(19) United States
(12) Patent Application Publication
(10) Pub. No.: US 2023/0242538 A1
(43) Pub. Date: Aug. 3, 2023

(54) PanK Modulators and Methods Using Same

(71) Applicant: YALE UNIVERSITY, New Haven, CT (US)

(72) Inventors: Choukri Ben Mamoun, New Haven, CT (US); Peter Gareiss, New Haven, CT (US); Jose Thekkiniath, New Haven, CT (US); Denton Hoyer, New Haven, CT (US); Yulia Surovtseva, New Haven, CT (US); William Hungerford, New Haven, CT (US); Mark Plummer, New Haven, CT (US); Antonella Stroppa, New Haven, CT (US); Joy Chiu, New Haven, IL (US)

(21) Appl. No.: 18/002,710

(22) PCT Filed: Jun. 23, 2021
(86) PCT No.: PCT/US21/38583

§ 371 Date: Dec. 21, 2022

Related U.S. Application Data
(60) Provisional application No. 63/043,534, filed on Jun. 24, 2020.

(51) Int. Cl. C07D 487/04; A61P 31/10; A61K 31/7048; A61K 45/06; A61K 31/519; A61K 31/5377

(52) U.S. Cl. CPC C07D 487/04; A61P 31/10; A61K 31/7048; A61K 45/06; A61K 31/519; A61K 31/5377

(57) ABSTRACT

The disclosure relates, in certain aspects, to the discovery of certain compounds that are useful to treat and/or prevent fungal infections in a subject. The disclosure further relates, in certain aspects, to the discovery of certain compounds that are useful are useful for treating, ameliorating, and/or preventing diseases or disorders associated with reduced and/or deficient PanK activity in a subject. In certain embodiments, the disease or disorder comprises a neurodegenerative disease, such as but not limited to pantothenate kinase-associated neurodegeneration (PKAN).

### CROSS-REFERENCE TO RELATED APPLICATIONS

This application claims priority under 35 U.S.C. § 119(e) to U.S. Provisional Patent Application No. 63/043,534, filed Jun. 24, 2020, the entire contents of which are incorporated herein by reference.

### BACKGROUND

In all living organisms, coenzyme A (CoA) plays a fundamental role in cellular metabolism, including synthesis and oxidation of fatty acids, and oxidation of pyruvate in the citric acid cycle. Its unique chemical structure, with a reactive thiol group and a nucleotide moiety, allows it to serve as a cofactor in critical biochemical and regulatory functions through activation of carboxylic acids and production of thioester derivatives.

In the yeast Saccharomyces cerevisiae, CoA is synthesized from vitamin B5 (pantothenic acid) either imported into the cell via the Fen2p pantothenate transporter or produced de novo in the cell from β-alanine and pantoate catalyzed by the enzyme pantothenate synthase Pan6p. Pantothenic acid utilization is absolutely essential for yeast survival as a double mutant lacking both the FEN2 and PANG genes is inviable. The first step in pantothenate utilization is its phosphorylation to 4′-phosphopantothenate by the pantothenate kinase (PanK) Cab1p, encoded by a single copy gene CAB1. This gene is required for yeast viability, since deletion of CAB1 results in cell death. A mutant cab1^{G351S}, which produces a kinase enzyme with glycine 351 substituted to serine, was found to be viable at 30° C. but is completely inviable at 37° C. Biochemical assays using purified wild type and mutated Cab1p enzymes showed that the G351S mutation results in 67˜94% loss of enzyme activity. The pantothenate analog α-PanAm inhibits yeast growth in a pantothenic acid dose-dependent manner. The MIC₅₀ of the compound was found to be about 4 times higher in medium supplemented with 1 μM pantothenic acid (MIC₅₀˜7.6 μg/mL) compared to medium with 100 nM pantothenic acid (MIC₅₀˜1.9 μg/mL). In vitro pantothenate phosphorylation assays and mass spectrometry analysis showed that α-PanAm is also phosphorylated by Cab1p and acts as a competitor of pantothenic acid at the enzyme catalytic site. α-PanAm has also been proposed to act on downstream steps in CoA biosynthesis and ultimately exerts its activity by reducing cellular CoA levels. In fungi, acetylation of CoA by acetyl-CoA synthetases generates acetyl-CoA, a key node in multiple metabolic and cellular processes including the synthesis of ergosterol, an essential component of the plasma and mitochondrial membranes. Ergosterol serves fundamental cellular functions such as maintenance of membrane fluidity, permeability to nutrients and solutes and response to environmental stresses. The ergosterol biosynthesis pathway from acetyl-CoA involves 24 known Erg enzymes and can be divided into two main sub-pathways, the mevalonate route and the squalene-to-ergosterol route (also known as the distal ergosterol biosynthesis route) (FIG. 8). The mevalonate route produces farnesyl pyrophosphate, which in the ERG biosynthesis pathway serves as an intermediate in the synthesis of the triterpene (C30) squalene, whereas the distal route produces ergosterol from squalene and is the main target of the antifungal drug classes of allylamines, azoles, morpholines, and polyenes (FIG. 8). Some of the ergosterol biosynthesis enzymes in the distal route can also catalyze alternative reactions to produce non-physiological sterol intermediates, some of which have been shown to inhibit fungal growth. This ability becomes relevant in cases of inhibition of enzymes in the pathway that cause accumulation of toxic steroidal substrates, which can then be metabolized through these alternative reactions. One of the first rate-limiting steps in the distal pathway is oxygenation of squalene to squalene epoxide catalyzed by squalene epoxidase, Erglp. This enzyme is the target of the major allylamine-type antifungal drug terbinafine. Inhibition of Erglp by terbinafine results in accumulation of squalene in the cell, which impairs fungal membrane function.

Fungal diseases are a major global health problem and are particularly threatening as opportunistic infections in immunosuppressed individuals such as AIDS and cancer patients. Despite major advances in understanding the biology of fungi and in antifungal drug discovery, fungal infections continue to cause significant mortality and morbidity worldwide. Commonly used antifungal drugs include azoles, echinocandins, polyenes, and allylamines. However, due to widespread resistance to some of these drugs and their lack of efficacy against a diverse array of pathogens such as Candida albicans and Aspergillus fumigatus, both new drugs and alternative strategies to modulate the efficacy and safety of current drugs and reverse resistance are urgently needed.

In mammals (including humans) there are at least four closely related active PanK isoforms—PanK1a, PanK1, PanK2, and PanK3—which are encoded by three genes. The PanKs regulate cellular CoA through feedback inhibition of the enzyme activity by CoA itself or CoA thioesters, and each isoform responds to inhibition with a distinct sensitivity. The PanK isoform expression profiles differ among individual cell types, tissues, and organs, and the relative abundance of one or more isoforms determines the respective CoA levels.

Inborn errors of CoA biosynthesis lead to neurodegenerative disorders in humans. Pantothenate kinase-associated neurodegeneration (PKAN; formerly called Hallervorden-Spatz syndrome) is a rare genetic neurodegenerative disease caused by mutations in the gene coding for PanK2 (which is localized to the mitochondria). PanK2 inactivation results in significantly decreased CoA levels in the cells, thereby reducing neuronal metabolism and function. Classic PKAN develops in the first 10 years of life, starting around age 3, and leading to severe neurodegeneration, with damage to brain, retina, and testis, and ultimately premature death.

There is thus a need in the art for the identification of novel compounds that can be used to treat and/or prevent fungal infections. There is also a need in the art to identify compounds, compositions, and methods useful for treating, ameliorating, and/or preventing neurodegenerative diseases and/or disorders such as PKAN. The present disclosure addresses these needs.

### BRIEF SUMMARY OF THE DISCLOSURE

In one aspect, the present disclosure relates to a pharmaceutical composition comprising at least one pharmaceutically acceptable carrier and at least one compound of formula (Ia) or (Ib), or a salt, solvate, isotopically labelled derivative, stereoisomer, tautomer, or geometric isomer thereof, and any mixtures thereof:

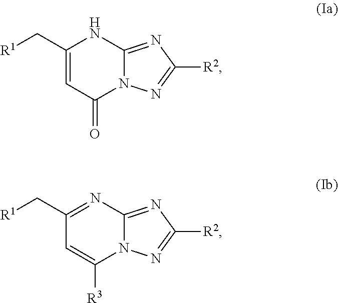

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein. In certain embodiments, the pharmaceutical composition comprises an agent useful for treating, ameliorating, and/or preventing a fungal infection in a mammal. In certain embodiments, the pharmaceutical composition comprises which further comprises an agent useful for treating, ameliorating, and/or preventing a neurological disease or disorder and/or an Acyl-CoA dehydrogenation deficiency in a mammal.

In another aspect, the present disclosure relates to a compound of formula (Ia) or (Ib), or a salt, solvate, isotopically labelled derivative, stereoisomer, tautomer, or geometric isomer thereof, and any mixtures thereof:

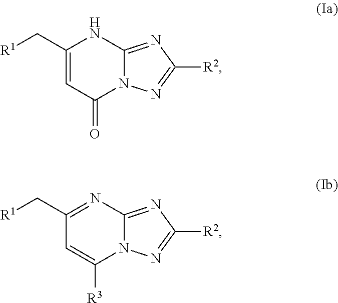

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein.

In yet another aspect, the present disclosure relates to a method of inhibiting pantothenate kinase (PanK) in a fungus, wherein the method comprises contacting the fungus with a compound of formula (Ia) or (Ib), or a salt, solvate, isotopically labelled derivative, stereoisomer, tautomer or geometric isomer thereof and any mixtures thereof:

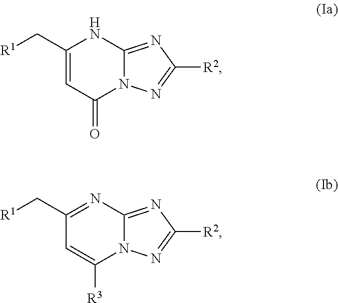

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein.

In yet another aspect, the present disclosure relates to a method of treating, ameliorating, and/or preventing a fungal infection in a subject, wherein the method comprises administering to the subject a therapeutically effective amount of a compound of formula (Ia) or (Ib), or a salt, solvate, isotopically labelled derivative, stereoisomer, tautomer, or geometric isomer thereof, and any mixtures thereof:

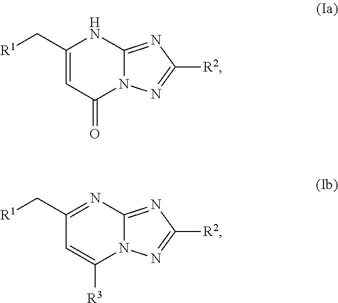

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein.

In yet another aspect, the present disclosure relates to a method of activating pantothenate kinase (PanK) in a human cell, wherein the method comprises contacting the human cell with a compound of formula (Ia) or (Ib), or a salt, solvate, isotopically labelled derivative, stereoisomer, tautomer, or geometric isomer thereof, and any mixtures thereof.

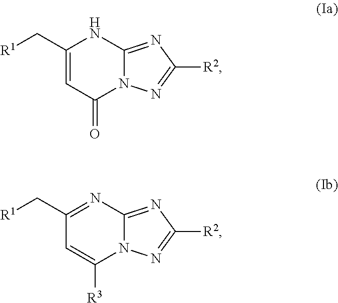

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein.

In yet another aspect, the present disclosure relates to a method of treating, ameliorating, and/or preventing a disease or disorder associated with reduced and/or deficient PanK activity, and/or a disease or disorder associated with reduced cellular free CoA levels, in a human subject, wherein the method comprises administering to the subject a therapeutically effective amount of a compound of formula (Ia) or (Ib), or a salt, solvate, isotopically labelled derivative, stereoisomer, tautomer, or geometric isomer thereof, and any mixtures thereof.

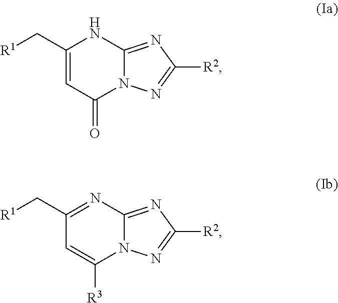

wherein R¹, R², and R³ are defined elsewhere herein. In certain embodiments, the compound of formula (Ia) or (Ib), or salt, solvate, isotopically labelled derivative, stereoisomer, tautomer, or geometric isomer thereof, and any mixtures thereof is excluded from certain compounds described elsewhere herein.

### BRIEF DESCRIPTION OF THE DRAWINGS

The following detailed description of selected embodiments of the disclosure will be better understood when read in conjunction with the appended drawings. For the purpose of illustrating the disclosure, non-limiting embodiments are shown in the drawings. It should be understood, however, that the disclosure is not limited to the precise arrangements and instrumentalities of the embodiments shown in the drawings.

FIGS. 1A-1C illustrate the finding that reduced pantothenate activity results in altered sensitivity to antifungals. FIG. 1A: WT and cab1^{ts} cells were spotted on YPD agar plates at cell densities ranging between 10³ and 10⁶ cells and incubated at 30° C. and 37° C. Images were collected 48 hours post-inoculation. FIG. 1B: Pantothenate kinase activity of wild type Cab1p and Cab1^{G351S}p measured using the Kinase Glo assay. *p-value<0.0001. FIG. 1C: WT and cab1^{ts} cells were spotted on YPD agar plates at limiting dilution, with cell densities ranging between 10⁶ cells/spot and 10¹ cells/spot. All plates were incubated at 30° C. in the absence or presence of 10 ng/mL amorolfine, 1 μg/mL amphotericin B, 10 μg/mL fluconazole, or 10 μg/mL terbinafine. All images were collected 48 hours post-onoculation.

FIGS. 2A-2H illustrate relationship between pantothenate utilization and yeast susceptibility to terbinafine. FIGS. 2A-2F: Growth curves of WT and cab1^{ts} cells without or with 160 μg/mL terbinafine (terb) in liquid minimal medium supplemented with varying concentrations of pantothenic acid. FIG. 2G: Relative times for treated cells to reach mid-log phase compared to untreated cells, calculated from FIG. 2A. Time for untreated cells to grow to mid-log was normalized to 1, and is represented by the dashed line. Comparing the WT to cab1^{ts} growths at each PA (pantothenic acid) concentration provides significant results. *p-value<0.0001; **p value<0.0001; ***p-value<0.0001. FIG. 2H: Isobologram of the interaction between terbinafine and the pantothenate analog α-PanAm (n=2). The solid line represents the theoretical curve of an additive effect. Data above the solid line (dashed curve) shows antagonism between the two compounds.

FIGS. 3A-3H illustrate relationship between pantothenate utilization and yeast susceptibility to amphotericin B. FIGS. 3A-3F: Growth curves of WT and cab1^{ts} cells without or with 1 μg/mL amphotericin B (amphB) in liquid minimal medium supplemented with varying concentrations of pantothenic acid. FIG. 3G: Relative times for treated cells to reach mid-log phase compared to untreated cells, calculated from FIG. 3A. Time for untreated cells to grow to mid-log was normalized to 1, and is represented by the dashed line. FIG. 3H: Isobologram of the interaction between amphotericin B and the pantothenate analog α-PanAm (n=2). The solid line represents the theoretical curve of an additive effect. Data below the solid line (dashed curve) shows synergism between the two compounds.

FIGS. 4A-4D illustrate the effect of altered pantothenate phosphorylation on cellular sterol levels and sensitivity to terbinafine. FIG. 4A: Most prominent ergosterol precursors in the ergosterol biosynthetic pathway were chosen for analysis in both WT and cab1^{ts} cells. FIG. 4B: Levels of squalene, lanosterol and ergosterol in WT cells were normalized to 1 and are represented by the dashed line on the graph. Bars denote the fold change in the relative level of each sterol in cab1^{ts} cells compared to wild type. *p-value 0.0088; **p-value 0.0185; ***p-value 0.0009. FIG. 4C: Growth of WT cells in defined liquid media with 100 μM PA, supplemented with squalene, terbinafine (terb), or both (n=3). FIG. 4D: Relative times of treated cells to grow to mid-log phase compared to untreated cells, calculated from values in FIG. 4C. Untreated cells were normalized to 1. *p-value 0.0017, **p-value 0.0002, ***p-value<0.0001.

FIGS. 5A-5B illustrate the effect of altered pantothenate phosphorylation on CAB and ERG gene transcription. FIG. 5A: Heatmap (log 2 scale) showing changes in the expression levels of two transcription factors for the ergosterol biosynthesis pathway, CAB genes, and ERG genes between wild type and cab1^{ts} cells grown in minimal media supplemented with 100 μM pantothenic acid. Two independent replicates of the wild type strain are represented by the left two columns, and two replicates of the cab1^{ts} strain are represented by the right two columns. FIG. 5B: Fold change of gene expression levels (FPKM) between wild type and cab1^{ts} strains (n=2). For each gene, the level in the wild type was set to 1 to normalize the data. The bars show relative fold change of cab1^{ts} cells to wild type for each gene. The relative FPKM levels of the ACT1 gene are shown for control. *p-value 0.0011, **p-value 0.0013, ***p-value 0.0003, ****p-value 0.0004.

FIG. 6 illustrates a model of the control mode of ergosterol biosynthesis and sensitivity to antifungal by CoA biosynthesis pathway. Inhibition or reduction of Cab1p activity through genetic mutation (use of cab1^{ts} mutant strain) or enzyme inhibition (addition of low-molecular inhibitor α-PanAm) results in reduced sensitivity to terbinafine or increased sensitivity to amphotericin B. Conversely, activation of Cab1p through supplementation with exogenous pantothenate or a surrogate for Cab1p activation by the addition of squalene results in increased sensitivity to terbinafine and reduced sensitivity to amphotericin B.

FIG. 7 illustrates simplified Coenzyme A and ergosterol biosynthesis pathways in yeast. Schematic representation of key intermediates in the metabolic pathways for CoA and ergosterol biosynthesis in the yeast S. cerevisiae. The CoA biosynthesis and ergosterol biosynthesis pathways are each boxed to demonstrate their separate treatment and lack of link in the current literature. The red arrow represents certain results discussed herein, demonstrating the regulation that the CoA biosynthesis pathway exerts over the ergosterol biosynthesis pathway. Solid arrows represent single enzymatic steps between intermediates. Dashed arrows indicate multiple steps between intermediates. Further detailed enzymatic steps involved in these processes are shown in FIG. 8.

FIG. 8 illustrates a detailed metabolic pathway from pantothenate to ergosterol. The pathway is divided into three sub-pathways: the CoA biosynthesis pathway from pantothenate (outlined in blue), the mevalonate pathway (outlined in green), and the distal ergosterol biosynthetic pathway (outlined in yellow). Solid arrows represent single enzymatic steps. Dashed arrows indicate multiple steps, catalyzed by the proteins in the order they are listed, from one intermediate to the next. Drug classes, individual drugs and their site of action are shown in red.

FIG. 9 illustrates changes in global yeast gene transcription between WT and cab1^{ts} cells. Both strains were grown in minimal media supplemented with 100 μM PA. The two independent replicates of WT cell mRNA are represented by the left two columns, and the two replicates of cab1^{ts} cell mRNA are represented by the right two columns. Genes were selected as significantly upregulated or downregulated between the WT and cab1^{ts} strains if q-values were less than 0.05 for the log 2 change between the two samples. 22 genes were found to be significantly upregulated and 38 genes significantly downregulated in cab1^{t}s cells compared to wild type cells. It was found that globally, maltose fermentation (MAL11, MAL12, MAL31, MAL32) was upregulated in the cab1^{ts} mutant. Additionally, there was downregulation of phosphate import into the cab1^{ts} cell (PH011, PH012, PH084, PH089), modulation of amino acid anabolic and catabolic pathways (MET1 7, LEU1, CAR2), and systematic downregulation of genes of genes transcriptionally regulated by factors involved in pleiotropic drug resistance (CIS1, PDR5, regulated by transcription factors Pdrlp, Yrrlp).

FIGS. 10A-10D illustrates the effect of altered pantothenate phosphorylation on genes regulating sterol trafficking. Fold change of gene expression levels (FPKM) between wild type and cab1^{t}s strains (n=2) grown in minimal medium supplemented with 100 μM PA. For each gene, the level in the wild type was set to 1 to normalize the data. The bars show relative fold change of cab1^{ts} cells to wild type for each gene. FIG. 10A: Genes regulating sterol uptake. *p-value 0.0198, **p-value 0.0052. FIG. 10B: Genes regulating sterol storage. *p-value 0.0266, **p-value 0.0189, ***p-value 0.0482. FIG. 10C: Genes regulating sterol transport between organelle membranes. *p-value 0.0195. FIG. 10D: Genes regulating exogenous sterol uptake. *p-value 0.0085.

### DETAILED DESCRIPTION

The disclosure relates, in certain aspects, to the identification of certain compounds that are useful to treat, ameliorate, and/or prevent fungal infections in a subject. In certain embodiments, the compounds of the disclosure have synergistic effects against fungi when used in combination with other antifungal agents, such as but not limited to agents that disrupt synthesis and/or activity of ergosterol, such as but not limited to amphotericin B.

The disclosure further relates, in certain aspects, to the identification of certain compounds that are useful for treating, ameliorating, and/or preventing diseases or disorders associated with reduced and/or deficient PanK activity in a human subject. In certain embodiments, the disease or disorder comprises a neurodegenerative disease or disorder. In certain embodiments, the neurodegenerative disease or disorder comprises pantothenate kinase-associated neurodegeneration (PKAN).

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of 0, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “neurodegenerative disease or disorder” refers to a disease or disorder selected from Pantothenate kinase-associated neurodegeneration (PKAN), Alzheimer's disease, Parkinson's disease, Huntington's disease, Amyotrophic lateral sclerosis (ALS), Dementia Ataxia telangiectasia, Autosomal dominant cerebellar ataxia, Batten disease, Corticobasal degeneration, Creutzfeldt-Jakob disease, Fatal familial insomnia, Hereditary motor and sensory neuropathy with proximal dominance, Infantile Refsum disease, JUNQ and IPOD, Locomotor ataxia, Lyme disease, Machado-Joseph disease, Mental retardation and microcephaly with pontine and cerebellar hypoplasia, Multiple system atrophy, Neuroacanthocytosis, Niemann-Pick disease, Pontocerebellar hypoplasia, Refsum disease, Sandhoff disease, Shy-Drager syndrome, Spinocerebellar ataxia, Subacute combined degeneration of spinal cord, Subacute sclerosing panencephalitis, Tabes dorsalis, Tay-Sachs disease, Toxic encephalopathy, and Wobbly hedgehog syndrome. Selected neurodegenerative diseases or disorders in the context of the present disclosure are Pantothenate kinase-associated neurodegeneration (PKAN), Alzheimer's disease, Parkinson's disease, Huntington's disease, Amyotrophic lateral sclerosis (ALS), Dementia. In certain embodiments, the disease or disorder is Pantothenate kinase-associated neurodegeneration (PKAN).

As used herein, the term “organic aciduria” refers to a metabolic disorder in which normal amino acid metabolism, particularly branched-chain amino acids (e.g., isoleucine, leucine, and/or valine) is disrupted causing a buildup of acids that are usually not present in the cell. In certain embodiments, the organic aciduris comprises methylmalonic acidemia, propionic acidemia, isovaleric acidemia, and/or maple syrup urine disease.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR′, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, —N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, —C(═O)N(R^{b})(R^{b}), —C(═O)OR^{b}, —OC(═O)R^{b}, —SR^{b}, —S(═O)R^{b}, —S(═O)₂R^{b}, and —N(R^{b})S(═O)₂R^{b}, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, the alkyl, alkenyl, alkynyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, oxadiazolyl (including 1,2,3-, 1,2,4-, 1,2,5-, and 1,3,4-oxadiazole), and triazolyl (such as 1,2,3-triazolyl and 1,2,4-triazolyl).

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxazolidinyl, azetidinyl, and the corresponding oxo analogues (where a methylene ring group is replaced with a carbonyl) thereof.

Unless otherwise noted, when two substituents are taken together to form a ring having a specified number of ring atoms (e.g., R′ and R″ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

Compounds

The disclosure includes a compound described herein, or a salt, solvate, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

In certain embodiments, the compound is a compound of formula (Ia) or (Ib), or a salt, solvate, isotopically labelled derivative, stereoisomer, tautomer, or geometric isomer thereof, and any mixtures thereof:

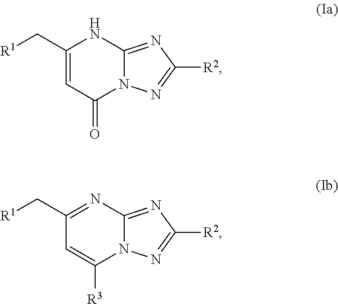

wherein in (Ia) or (Ib):

R¹ is selected from the group consisting of H, —NR^{4a}R^{4b}, —NR^{4c} (optionally substituted phenyl), and optionally substituted N-linked heterocyclyl;

R² is selected from the group consisting of optionally substituted phenyl and —N(R^{4c})—(CH₂)₁₋₃— (optionally substituted phenyl);

R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})(CH₂)₁₋₃— optionally substituted phenyl), and —C(═O)N(R^{4h})—(CH₂)₁₋₃-(optionally substituted heteroaryl);

each occurrence of R^{4a}-R^{4h} is independently selected from the group consisting of H, optionally substituted C₁-C₆ alkyl, and optionally substituted C₃-C₈ cycloalkyl; and each occurrence of alkyl, cycloalkyl, phenyl, heterocyclyl, and heteroaryl is optionally independently substituted with at least one of C₁-C₆ alkyl, C₃-C₈ cycloalkyl, halogen, cyano (—CN), —OR^{a}, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a}) S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form a C₃-C₈ heterocyclyl.

In certain embodiments, le is H. In certain embodiments, R¹ is —NR^{4a}, R_{4b}. In certain embodiments, le is —NR^{4c} (optionally substituted phenyl). In certain embodiments, le is optionally substituted N-linked heterocyclyl.

In certain embodiments, R² is optionally substituted phenyl. In certain embodiments, R² is —N(R^{4c})—(CH₂)₁₋₃-(optionally substituted phenyl);

In certain embodiments, R³ is F. In certain embodiments, R³ is C₁. In certain embodiments, R³ is Br. In certain embodiments, R³ is I. In certain embodiments, R³ is —OR^{4d}. In certain embodiments, R³ is —NR^{4d}R^{4e}. In certain embodiments, R³ is —C(═O)OR^{4d}. In certain embodiments, R³ is —C(═O)NR^{4e}R^{4f}. In certain embodiments, R³ is —C(═O)N(R^{4g})—(CH₂)₁₋₃-optionally substituted phenyl). In certain embodiments, R³ is —C(═O)N(R^{4h})—(CH₂)₁₋₃-(optionally substituted heteroaryl).

In certain embodiments, R^{4a} is H. In certain embodiments, R^{4a} is optionally substituted C₁-C₆ alkyl. In certain embodiments, R^{4a} is optionally substituted C₃-C₈ cycloalkyl.

In certain embodiments, R^{4b} is H. In certain embodiments, R^{4a} is optionally substituted C₁-C₆ alkyl. In certain embodiments, R^{4b} is optionally substituted C₃-C₈ cycloalkyl.

In certain embodiments, R^{4c} is H. In certain embodiments, R^{4c} is optionally substituted C₁-C₆ alkyl. In certain embodiments, R^{4c} is optionally substituted C₃-C₈ cycloalkyl.

In certain embodiments, R^{4d} is H. In certain embodiments, R^{4d} is optionally substituted C₁-C₆ alkyl. In certain embodiments, R^{4d} is optionally substituted C₃-C₈ cycloalkyl.

In certain embodiments, R^{4e} is H. In certain embodiments, R^{4a} is optionally substituted C₁-C₆ alkyl. In certain embodiments, R^{4e} is optionally substituted C₃-C₈ cycloalkyl.

In certain embodiments, R^{4f} is H. In certain embodiments, R^{4f} is optionally substituted C₁-C₆ alkyl. In certain embodiments, R^{4f} is optionally substituted C₃-C₈ cycloalkyl.

In certain embodiments, R^{4g} is H. In certain embodiments, R^{4g} is optionally substituted C₁-C₆ alkyl. In certain embodiments, R^{4g} is optionally substituted C₃-C₈ cycloalkyl.

In certain embodiments, R^{4h} is H. In certain embodiments, R^{4h} is optionally substituted C₁-C₆ alkyl. In certain embodiments, R^{4h} is optionally substituted C₃-C₈ cycloalkyl.

In certain embodiments, le is selected from the group consisting of H, —NR^{4a}R^{4b}, and optionally substituted N-linked heterocyclyl.

In certain embodiments, le is NR^{4c} (optionally substituted phenyl).

In certain embodiments, R³ is —NR_{4d}R^{4e}.

In certain embodiments, R³ is —C(═O)OR^{4d}.

In certain embodiments, each phenyl in R² is independently substituted with at least one C₂-C₈ alkyl. In other embodiments, each phenyl in R² is independently substituted at the ortho-position with at least one C₂-C₈ alkyl. In other embodiments, each phenyl in R² is independently substituted at the meta-position with at least one C₂-C₈ alkyl. In other embodiments, each phenyl in R² is independently substituted at the para-position with at least one C₂-C₈ alkyl.

In certain embodiments, the N-linked heterocyclyl is aziridinyl. In certain embodiments, the N-linked heterocyclyl is azetidinyl. In certain embodiments, the N-linked heterocyclyl is pyrrolidinyl. In certain embodiments, the N-linked heterocyclyl is pyrazolidinyl. In certain embodiments, the N-linked heterocyclyl is piperidinyl. In certain embodiments, the N-linked heterocyclyl is 1,2,3,6-tetrahydropyridinyl. In certain embodiments, the N-linked heterocyclyl is 1,4-dihydropyridinyl. In certain embodiments, the N-linked heterocyclyl is piperazinyl. In certain embodiments, the N-linked heterocyclyl is morpholinyl. In certain embodiments, the N-linked heterocyclyl is thiomorpholinyl. In certain embodiments, the N-linked heterocyclyl is homopiperazinyl. In certain embodiments, the N-linked heterocyclyl is or homopiperidinyl.

In certain embodiments, the heteroaryl is imidazolyl. In certain embodiments, the heteroaryl is pyridinyl. In certain embodiments, the heteroaryl is pyrimidinyl. In certain embodiments, the heteroaryl is pyrazinyl. In certain embodiments, the heteroaryl is thienyl. In certain embodiments, the heteroaryl is furyl. In certain embodiments, the heteroaryl is pyrrolyl. In certain embodiments, the heteroaryl is thiazolyl. In certain embodiments, the heteroaryl is oxazolyl. In certain embodiments, the heteroaryl is pyrazolyl. In certain embodiments, the heteroaryl is isothiazolyl. In certain embodiments, the heteroaryl is 1,2,3-triazolyl. In certain embodiments, the heteroaryl is 1,2,4-triazolyl. In certain embodiments, the heteroaryl is 1,3,4-triazolyl. In certain embodiments, the heteroaryl is tetrazolyl. In certain embodiments, the heteroaryl is 1,2,3-thiadiazolyl. In certain embodiments, the heteroaryl is 1,2,3-oxadiazolyl. In certain embodiments, the heteroaryl is 1,3,4-thiadiazolyl. In certain embodiments, the heteroaryl is 1,3,4-oxadiazolyl.

Non-limiting examples of the compound of formula (Ia) or (Ib) include:

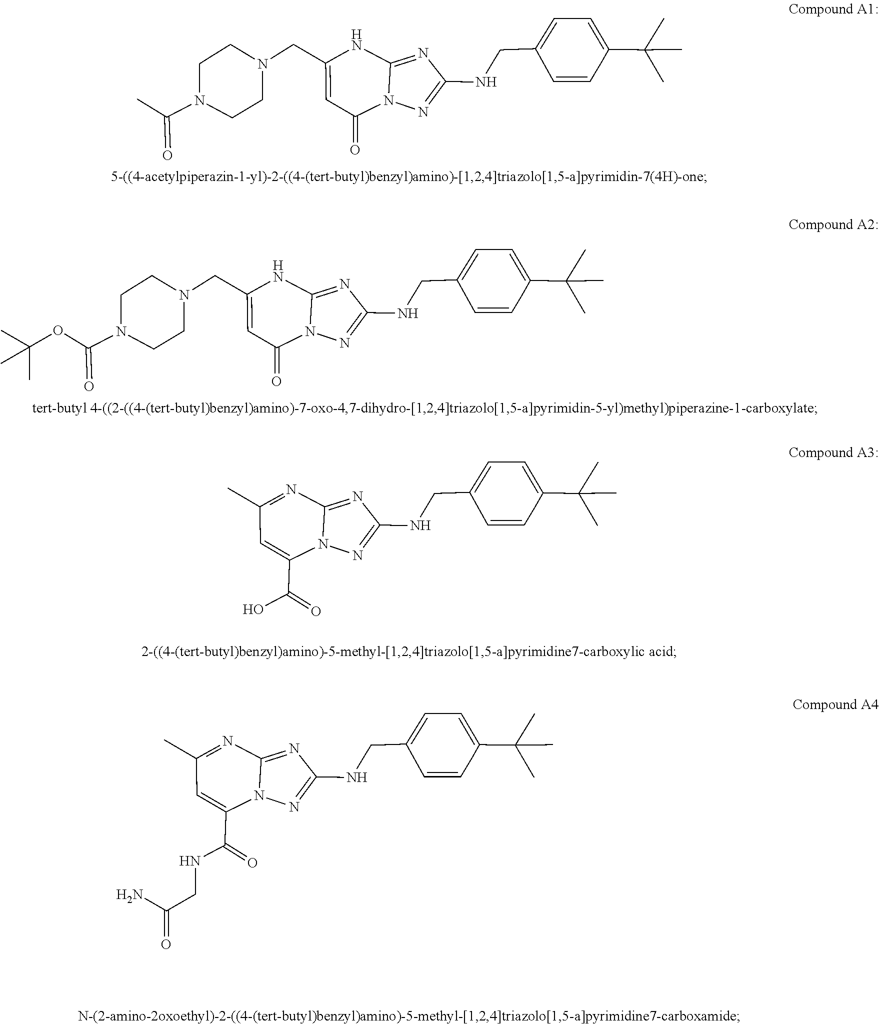

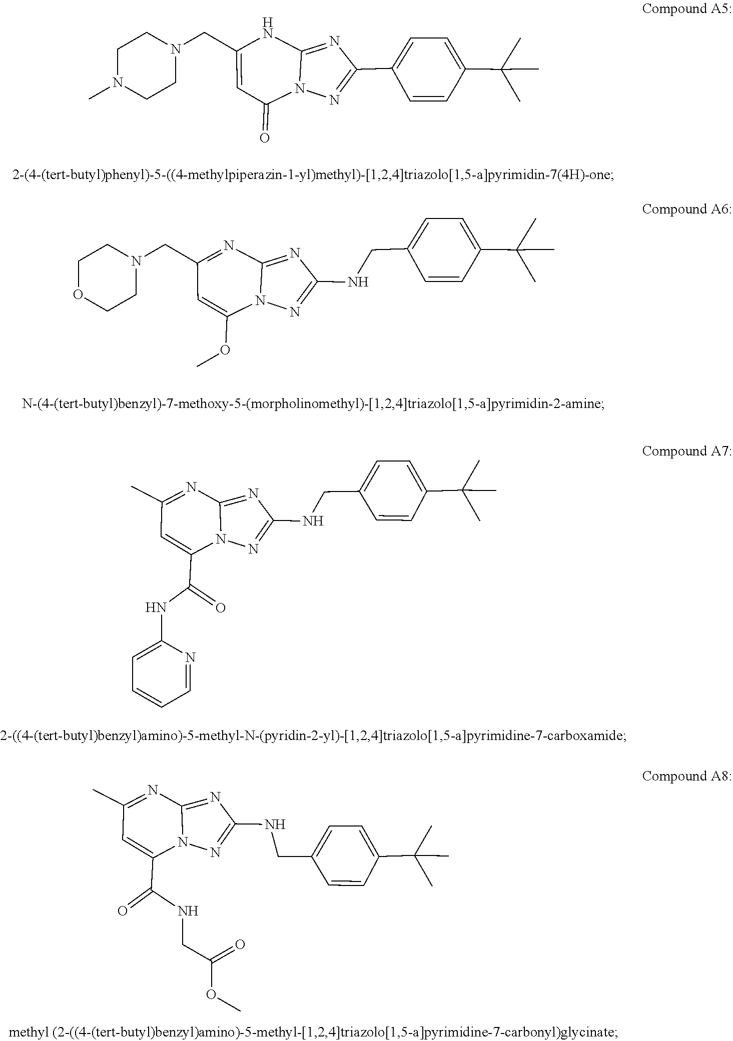

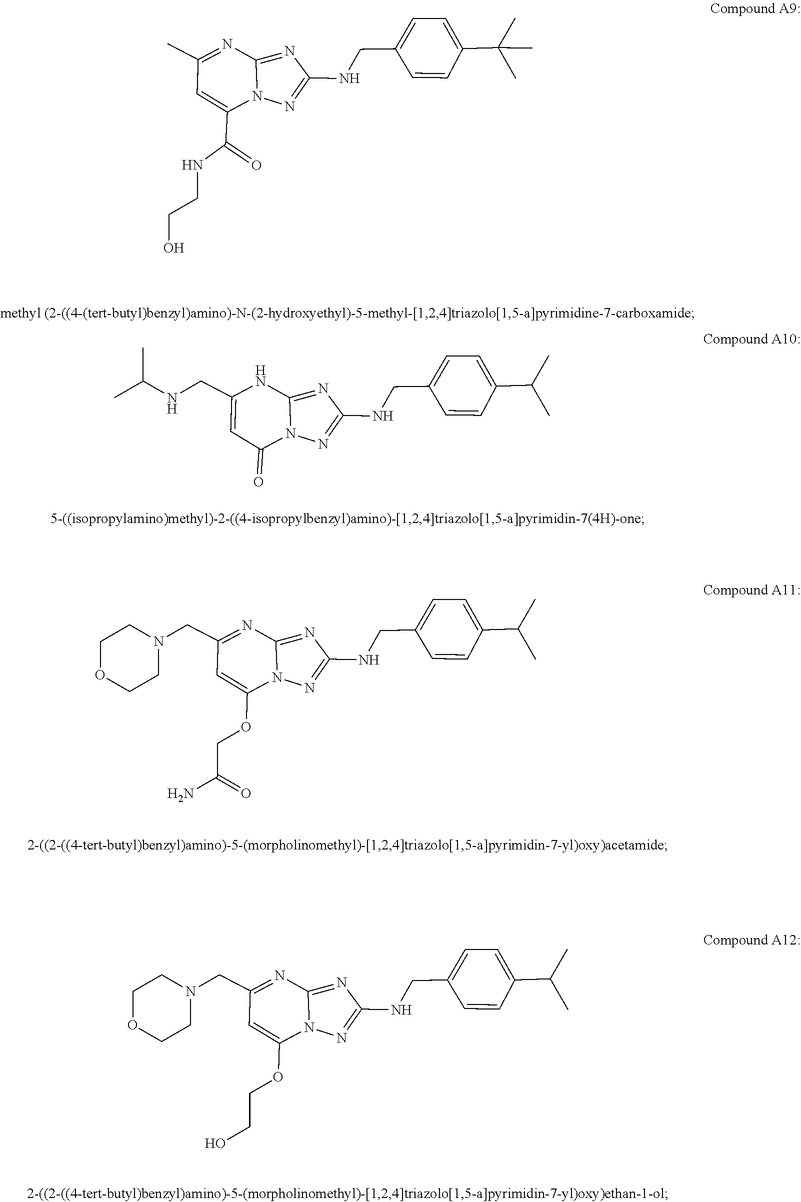

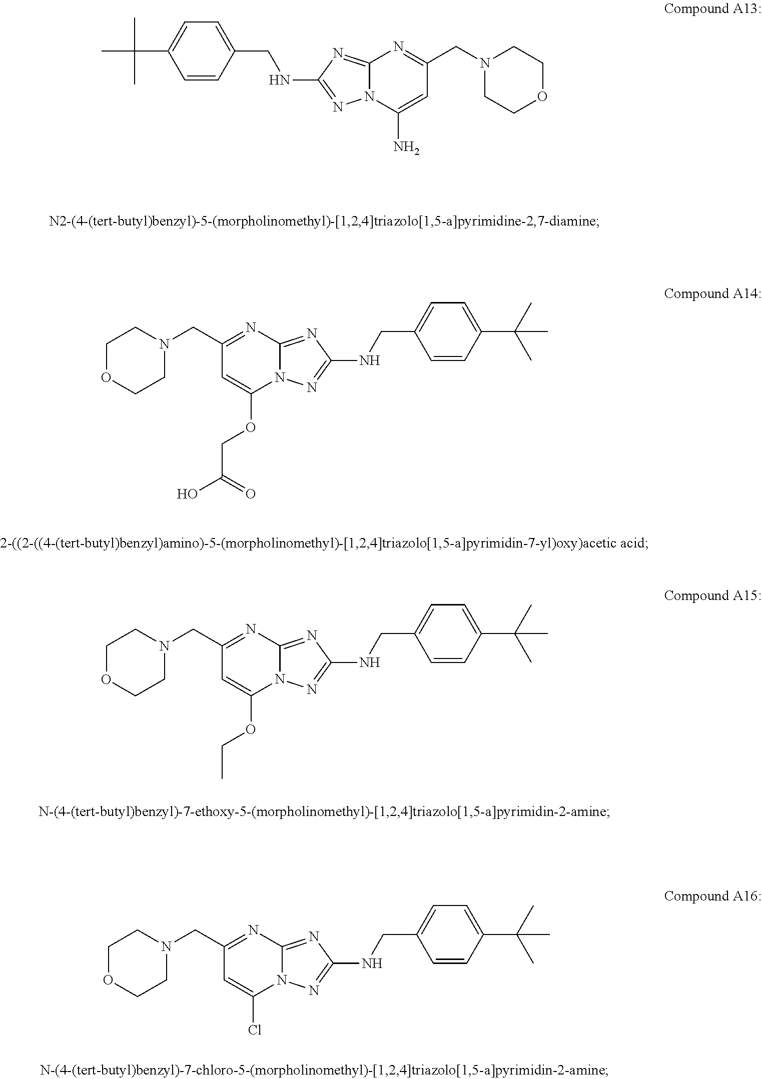 [SMILES: CC(C)(C)c1ccc(CNc2nc3nc(CN4CCOCC4)cc(N)n3n2)cc1 ; CC(C)(C)c1ccc(CNc2nc3nc(CN4CCOCC4)cc(OCC(=O)O)n3n2)cc1 ; CCOc1cc(CN2CCOCC2)nc2nc(NCc3ccc(C(C)(C)C)cc3)nn12 ; CC(C)(C)c1ccc(CNc2nc3nc(CN4CCOCC4)cc(Cl)n3n2)cc1]

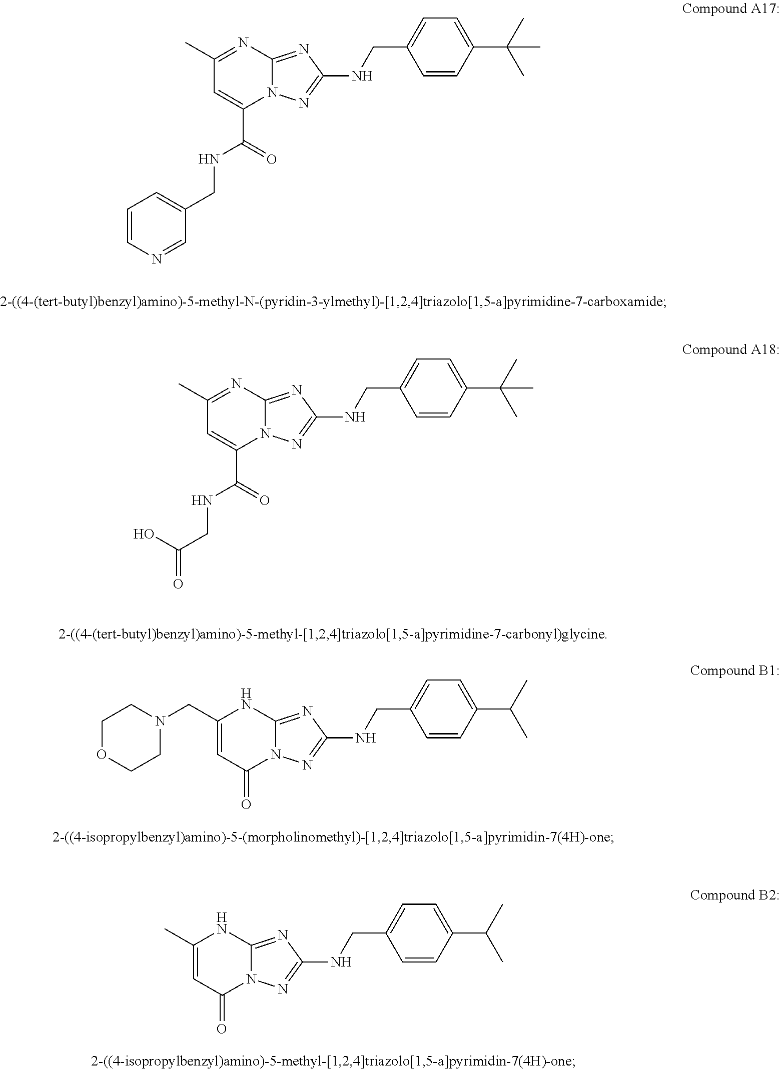

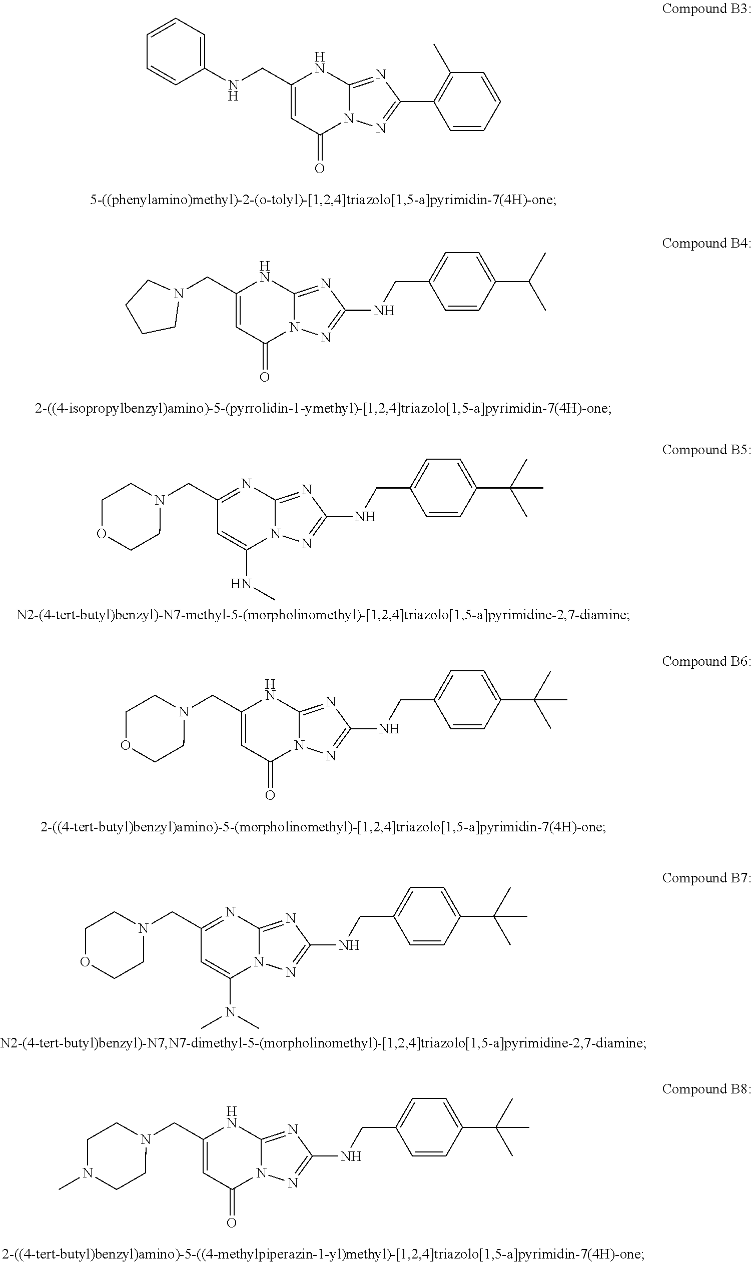

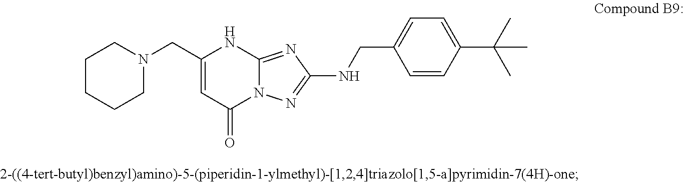 [SMILES: CC(C)(C)c1ccc(CNc2nc3[nH]c(CN4CCCCC4)cc(=O)n3n2)cc1]

or a salt, solvate, isotopically labelled derivative, stereoisomer, tautomer, or geometric isomer thereof, and any mixtures thereof.

In certain embodiments, the compound is not:
Compound A5: 2-(4-(tert-butyl)phenyl)-5-((4-methylpiperazin-1-yl)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof.

In certain embodiments, the compound is not:
Compound A10: 5-((isopropylamino)methyl)-2-((4-isopropylbenzyl)amino)-[1,2,4]triazolo[1,5-a]pyrimidin-7(4H)-one, or a salt, solvate, isotopically labelled derivative, stereoisomer, tautomer, or geometric isomer thereof, and any mixtures thereof.
   In certain embodiments, the compound is not:
Compound B1: 2-((4-isopropylbenzyl)amino)-5-(morpholinomethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof.

In certain embodiments, the compound is not:
Compound B2: 2-((4-isopropylbenzyl)amino)-5-methyl-[1,2,4]triazolo[1,5-a]pyrimidin-7(4H)-one, or a salt, solvate, isotopically labelled derivative, stereoisomer, tautomer, or geometric isomer thereof, and any mixtures thereof.

In certain embodiments, the compound is not:
Compound B3: 5-((phenylamino)methyl)-2-(o-tolyl)-[1,2,4]triazolo[1,5-a]pyrimidin-7(4H)-one, or a salt, solvate, isotopically labelled derivative, stereoisomer, tautomer, or geometric isomer thereof, and any mixtures thereof.

In certain embodiments, the compound is not:
Compound B4: 2-((4-isopropylbenzyl)amino)-5-(pyrrolidin-1-ylmethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof.

In certain embodiments, the compound is not 2-phenyl-5-((propylamino)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 5-((4-methylpiperidin-1-yl)methyl)-2-phenyl-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 5-((benzyl(methyl)amino)methyl)-2-phenyl-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 5-((benzyl(2-hydroxyethyl)amino)methyl)-2-phenyl-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(4-chlorophenyl)-5-((3,4-dihydroisoquinolin-2(1H)-yl)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 5-((2-methoxyphenyl)amino)methyl)-2-(p-tolyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(p-tolyl)-5-((m-tolylamino)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(p-tolyl)-5-((p-tolylamino)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(p-tolyl)-5-((o-tolylamino)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(4-methoxyphenyl)-5-(piperidin-1-ylmethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(4-methoxyphenyl)-5-(morpholinomethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 54(2,4-dimethoxyphenyl)amino)methyl)-2-(4-methoxyphenyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(4-(tert-butyl)phenyl)-5-((propylamino)methyl)[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-(3,4-dimethylphenyl)-5-((p-tolylamino)methyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 5-methyl-2-(phenylamino)-[1,2,4]triazolo[1,5-a]pyrimidin-7-ol, or a salt, solvate, isotopically labelled derivative, stereoisomer, tautomer, or geometric isomer thereof, and any mixtures thereof. In certain embodiments, the compound is not 5-methyl-2-phenyl-[1,2,4]triazolo[1,5-a]pyrimidin-7-ol, or a salt, solvate, isotopically labelled derivative, stereoisomer, tautomer, or geometric isomer thereof, and any mixtures thereof. In certain embodiments, the compound is not 2-(benzylamino)-5-(morpholinomethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not 2-((3,4-dimethoxybenzyl)amino)-5-(morpholinomethyl)-[1,2,4]triazolo[1,5-a]pyrimidin-7(4H)-one, or a salt, solvate, isotopically labelled derivative, stereoisomer, tautomer, or geometric isomer thereof, and any mixtures thereof. In certain embodiments, the compound is not methyl 2-((2-((4-(tert-butyl)benzyl)amino)-5-(morpholinomethyl)-[1,2,4]triazolo[1,5-a]pyrimidin-7-yl)oxy)acetate, or a salt, solvate, isotopically labelled derivative, stereoisomer, tautomer, or geometric isomer thereof, and any mixtures thereof. In certain embodiments, the compound is not 7-(benzyloxy)-N-(4-(tert-butyl)benzyl)-5-(morpholinomethyl)-[1,2,4]triazolo[1,5-a]pyrimidin-2-amine, or a salt, solvate, isotopically labelled derivative, stereoisomer, tautomer, or geometric isomer thereof, and any mixtures thereof. In certain embodiments, the compound is not 2-((2-((4-(tert-butyl)benzyl)amino)-5-(morpholinomethyl)-[1,2,4]triazolo[1,5-a]pyrimidin-7-yl)oxy)-N,N-dimethylacetamide.

In certain embodiments, the compound of the disclosure is any compound disclosed herein, or a salt, solvate, prodrug, isotopically labelled, stereoisomer, any mixture of stereoisomers, tautomer, and/or any mixture of tautomers thereof.

In certain embodiments, the compound is at least one selected from Table 1 or Table 2, or a salt, solvate, prodrug, isotopically labelled, stereoisomer, any mixture of stereoisomers, tautomer, and/or any mixture of tautomers thereof.

The compounds of the disclosure may possess one or more stereocenters, and each stereocenter may exist independently in either the (R)- or (S)-configuration. In certain embodiments, compounds described herein are present in optically active or racemic forms.

The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

Combination Therapies

In one aspect, the compounds of the disclosure are useful within the methods of the disclosure in combination with one or more additional agents useful for treating or preventing a disease or disorder contemplated herein. These additional agents may comprise compounds or compositions identified herein, or compounds (e.g., commercially available compounds) known to treat, prevent, or reduce the symptoms of a disease or disorder contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

Methods

The disclosure provides a method of inhibiting pantothenate kinase in a fungus. In certain embodiments, the method comprises contacting the fungus with a compound of the disclosure.

The disclosure further provides a method of treating or preventing a fungal infection in a subject. In certain embodiments, the method comprises administering to the subject a therapeutically effective amount of a compound of the disclosure. In other embodiments, the subject is further administered amphotericin B.

In certain embodiments, the fungus comprises at least one of Candida (such as but not limited to C. albicans, C. glabrata, C. auris, and C. parapsilosis), Aspergillus (such as but not limited to A. fumigatus and A. terreus), Histoplasma, Cryptococcus, and Mucor.

In certain embodiments, the compound of the disclosure is the only antifungal agent administered to the subject. In other embodiments, the compound of the disclosure is the only antifungal agent administered to the subject in a sufficient amount to treat or prevent the fungal infection. In yet other embodiments, the compound of the disclosure is administered to the subject in a pharmaceutically acceptable composition.

In certain embodiments, the compound of the disclosure and amphotericin B are co-formulated.

In certain embodiments, administration of the compound of the disclosure and amphotericin B results in less likely drug resistance occurrence in the subject as compared to an equivalent subject that is administered amphotericin B in the absence of the compound of the disclosure.

In certain embodiments, administration of the compound of the disclosure and amphotericin B allows for administration of an amount of the amphotericin B that is lower than the corresponding amount of amphotericin B that has to be administered in the absence of the compound of the disclosure to achieve equivalent treatment or prevention of the fungal infection.

In certain embodiments, the compound of the disclosure inhibits fungal PanK selectively over a human PanK enzyme.

In certain embodiments, the subject is a mammal. In other embodiments, the mammal is a human.

The disclosure provides a method of activating, and/or increasing the activity of, pantothenate kinase in a human cell. In certain embodiments, the method comprises contacting the human cell with a compound of the disclosure.

The disclosure further provides a method of treating, ameliorating, and/or preventing diseases or disorders associated with reduced and/or deficient PanK activity, and/or diseases or disorders associated with reduced cellular free CoA levels (such as, but not limited to, an organic aciduria) in a subject. In certain embodiments, the method comprises administering to the subject a therapeutically effective amount of a compound of the disclosure.

In certain embodiments, the disease or disorder comprises a neurodegenerative disease or disorder. In certain embodiments, the neurodegenerative disease or disorder comprises pantothenate kinase-associated neurodegeneration (PKAN).

In certain embodiments, the organic aciduria comprises methylmalonic acidemia, propionic acidemia, isovaleric acidemia, and/or maple syrup urine disease.

In certain embodiments, the compound of the disclosure is the only therapeutic agent administered to the subject. In other embodiments, the compound of the disclosure is the only therapeutic agent administered to the subject in a sufficient amount to treat or prevent the neurodegenerative disease or disorder. In yet other embodiments, the compound of the disclosure is administered to the subject in a pharmaceutically acceptable composition.

In certain embodiments, the subject is a mammal. In other embodiments, the mammal is a human.

Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

Materials & Methods

Yeast Strains:

Yeast strains (Saccharomyces cerevisiae) used in this study are JS91.15-23 (MATα his3 leu2 trp1 ura3) and JS91.14-24 (MATa ura3 his3 cab1ts) (Olzhausen, et al., 2009, Curr Genet 55, 163-173). Wild type and mutant strains were propagated either in YPD medium (2% bacto-peptone, 2% D-(+)-glucose and 1% yeast extract) or defined pantothenic acid-free (minimal) medium composed of Yeast Nitrogen Base (MP Biomedicals), supplemented with Complete Supplement Mixture (MP Biomedicals) and all vitamins except pantothenic acid. Where indicated, media were supplemented with appropriate concentrations of pantothenic acid.

Growth Assays on Solid and Liquid Media:

Spotting assay were performed as follows. Pre-cultures of WT and cab1^{ts} yeast strains were prepared in YPD medium overnight at 30° C. Cells were harvested, washed, and diluted to 10⁸ cells in 500 μL sterile water. Subsequent serial dilutions were made and 5 μL of cell suspensions were spotted on YPD agar plates lacking or supplemented with amorolfine 10 ng/mL, amphotericin B 1 μg/mL, fluconazole 10 μg/mL or terbinafine 10 μg/mL to achieve 10⁶, 10⁵, 10⁴, 10³, 10², and 10¹ cells per spot. Plates were incubated at 30° C. or 37° C. and imaged every 24 hours. For liquid assay in 96-well plate format, cells were pre-cultured overnight in liquid YPD medium at 30° C., washed three times in water and diluted to achieve 10⁴ cells per well in 150 μL of minimal medium supplemented with 1 μM, 10 μM or 100 μM pantothenic acid and either lacking or supplemented with terbinafine (160 μg/ml). All plates were incubated at 30° C. Optical density measurements were taken with a BioTek SynergyMx microplate reader (OD630) every 8 hours for a total of 96 hours. Relative time to mid-log phase was calculated by first graphing growth curves of the varying concentrations of terbinafine for each concentration of pantothenic acid, and then determining the value at which the untreated curve reached saturation, and dividing it by 2 to represent the mid-log value. Dividing the time to mid-log phase of treated cells by the time to mid-log phase of untreated gave the relative time to mid-log phase.

Pantothenate Kinase Activity Assay:

The activity of recombinant His6-tagged Cab1p and cab1^{G351S} was determined in vitro using the Kinase-Glo Plus Luminescent Kinase (Kinase-Glo) assay as described in Chiu, et al., 2017, Scientific Reports 7:14234. The assay measures the amount of ATP remaining in solution following the kinase reaction. Briefly, kinase buffer consisted of 100 mM Tris-HCl pH 7.4, 10 mM MgCl₂, 100 μM pantothenate (PA), 100 μM ATP, and 0.5 mg/mL γ-globulin. Kinase reactions were initiated with the addition of 500 ng of purified Cab1p or Cab1^{G351S} in elution buffer (10 mM Tris pH 7.4, NaCl 10 mM, 500 mM imidazole). Heat-inactivated (80° C. for 20 min) Cab1p or Cab were used as controls. Samples were incubated at room temperature for 1 hour. At the end of the incubation, an equal volume (10 μL) of Kinase-Glo reagent was added to each sample, the plates were further incubated for 5 min and luminescence was recorded using BioTek SynergyMx microplate reader (excitation wavelength 485; emission wavelength 528).

Drug-Drug Interactions and Isobologram Calculations:

Terbinafine-α-PanAm interactions were determined using liquid growth assays in 96-well plates using the checkerboard method as described in Orhan, et al., 2005, J. Clin. Microbiol. 43:140-143. The isobologram was produced by solving for the sum 50% fractional inhibitory concentration (ΣFIC₅₀) using the equation:

$$Σ F I C 50 = \frac{{MIC}_{50} of the drug combination}{{MIC}_{50} of drug A alone} + \frac{{MIC}_{50} of the drug combination}{{MIC}_{50} of drug B alone}$$

The data from the time-point where the 0 μg/mL terbinafine curve reached saturation was used to generate percent inhibition values, representing growth inhibition based on the concentration of terbinafine. This was done by dividing the value of interest by the untreated (0 μg/mL terbinafine) value, multiplying by 100 to get a percentage, and then subtracting that percentage from 100, representing 100% inhibition. The resulting values were used to create an inhibition curve, using the best fit function to generate an MIC₅₀. This was done for both terbinafine in α-PanAm and α-PanAm in terbinafine. The resulting MIC₅₀ values were plugged into the ΣFIC₅₀ equation, and the ΣFIC₅₀ values were used to generate the curve.

Sterol Analysis:

Four independent 25 mL cultures of WT and cab1^{ts} were prepared in minimal medium supplemented with 100 μM PA at a starting cell density of 10⁴ cells/mL and incubated at 30° C. until they reached mid-log phase (average OD630˜0.6). Cells were then washed once in PBS, weighed, and stored at −80° C. until used. Sterol pattern was determined by GC-MS as described in Muller, et al., 2018, Molecules 23. The quantification, managed with an external calibration with ergosterol for the detected sterols and squalene for the content of squalene, consists of six levels with concentrations up to 10 μg/mL. The base peak of each sterol TMS ether (squalene and cholestane excluded) were taken as a quantifier ion for calculating the peak areas for squalene m/z 69, internal standard (IS) cholestane m/z 217, lichesterol (ergosta-5,8,22-trien-3β-ol) m/z 363, ergosterol (ergosta-5,7,22-trien-3β-ol) m/z 363, ergosta-5,7-dien3β-ol) m/z 365, fecosterol (ergosta-379 7,24(28)-dien-3β-ol) m/z 343, episterol (ergosta-7,24(28)-dien-3β-ol) m/z 343, fungisterol (ergost-7-en-3β-ol) m/z 472, lanosterol (4,4,14-trimethylcholesta-8,24-dien-3β-ol) m/z 393, and T-MAS (4,4-dimethylcholesta-8,24-dien-3β-ol) m/z 379.

Transcriptomic Analysis:

RNAseq was performed to compare the transcriptional profiles of WT cells and the cab1^{ts} mutant grown in minimal medium with 100 μM pantothenic acid (conditions at which the strains showed equal growth). RNA was isolated from cultures of wild type and cab1^{ts} using the RiboPure™ RNA Purification Kit (Invitrogen) and RNAseq was performed by running the samples on a HiSeq2500 in high-output mode 1×75 (200M reads/lane). The reads were trimmed for quality, and aligned with the hg19 reference genome using TopHat2. The transcripts were assembled using cufflinks. The assembled transcripts were used to estimate transcript abundance and differential gene-expression using cuffdiff. The results were visualized using R (CRAN) and cummerbund.

Statistics:

Relative time of cab1ts cells to grow to midlog phase compared to WT cells in liquid media with terbinafine or amphotericin B treatment were analyzed by unpaired t tests with a value 0.05, resulting in two-tailed p-values. For relative time to midlog phase in 160 μg/mL terbinafine and 1 μM PA: p-value<0.0001, t-value=23.52, df=4, n=3; 160 μg/mL terbinafine and 10 μM PA: p-value<0.0001, t-value=43.43, df=4, n=3; 160 μg/mL terbinafine and 100 μM PA: p-value<0.0001, t-value=54.73, df=4, n=3. For relative time to midlog phase in 1 μg/mL amphotericin B and 10 μM PA: p-value=0.8034, t value=0.2386, df=2, n=3; 1 μg/mL amphotericin B and 100 μM PA: p-value=0.3505, t value=1.208, df=2, n=3.

Fold changes of the sterols squalene, lanosterol, and ergosterol were analyzed by paired t-tests with α value 0.05, resulting in two-tailed p-values. For fold change squalene vs. WT: p-value=0.0088, t-value=10.61, df=2, n=3. For fold change lanosterol vs. WT: p-value=0.0185, t-value=7.253, df=2, n=3. For fold change ergosterol vs. WT: p-value=0.0009, t-value=33.22, df=2, n=3.

Relative time of growth in liquid media for WT cells treated with squalene and/or terbinafine compared to untreated WT cells were analyzed by unpaired t-tests with a value 0.05, resulting in two-tailed p-values. For cells treated with 100 μg/mL squalene, 0 μg/mL terbinafine: p-value=0.0017, t-value=7.493, df=4, n=3. For cells treated with 0 μg/mL squalene, 80 μg/mL terbinafine: p-value=0.0002, t-value=12.64, df=4, n=3. For cells treated with 100 μg/mL squalene, 80 μg/mL terbinafine: p-value<0.0001, t value=52.56, df=4, n=3.

Comparison of RNA transcription between FPKM values of WT and cab1^{ts} cells was statistically analyzed by unpaired t-tests with a value 0.05, resulting in two-tailed p values. Results follow. ACT1: p-value=0.1823, t-value=2.009, df=2, n=2; UPC2: p value=0.9826, t-value=0.024, df=2, n=2; ECM22: p-value=0.4907, t-value=0.837, df=2, n=2; CAB1: p-value=0.2669, t-value=1.524, df=2, n=2; ERG1: p-value=0.0011, t value=29.99, df=2, n=2; ERG11: p-value=0.0013, t-value=28.21, df=2, n=2; ERG24: p value=0.1965, t-value=1.909, df=2, n=2; ERG28: p-value=0.0003, t-value=54.77, df=2, n=2; ERG2: p-value=0.0004, t-value=47.94, df=2, n=2; ERG4: p-value=0.1103, t value=2.755, df=2, n=2; DAN1: p-value=0.0198, t-value=7.008, df=2, n=2; DAN4: p value=0.0052, t-value=13.78, df=2, n=2; AUS1: p-value=0.3317, t-value=1.27, df=2, n=2; PDR11: p-value=0.2838, t-value=1.451, df=2, n=2; SUT1: p-value=0.8514, t value=0.2125, df=2, n=2; ARE1: p-value=0.0266, t-value=6.014, df=2, n=2; ARE2: p value=0.0189, t-value=7.17, df=2, n=2; NPC2: p-value=0.0482, t-value=4.388, df=2, n=2; YFT2: p-value=0.0609, t-value=3.866, df=2, n=2; OSH7: p-value=0.1107, t value=2.75, df=2, n=2; OSH1/SWH1: p-value=0.3304, t-value=1.275, df=2, n=2; OSH2: p-value=0.1786, t-value=2.037, df=2, n=2; OSH3: p-value=0.4847, t-value=0.8503, df=2, n=2; OSH4/KES1: p-value=0.0669, t-value=3.67, df=2, n=2; OSH5/HES1: p value=0.0195, t-value=7.063, df=2, n=2; OSH6: p-value=0.6636, t-value=0.5053, df=2, n=2; PRY1: p-value=0.071, t-value=3.536, df=2, n=2; PRY2: p-value=0.0604, t value=3.882, df=2, n=2; PRY3: p-value=0.0085, t-value=10.76, df=2, n=2.

### Example 1: Reduced Pantothenate Phosphorylation Results in Modulated Drug Susceptibility

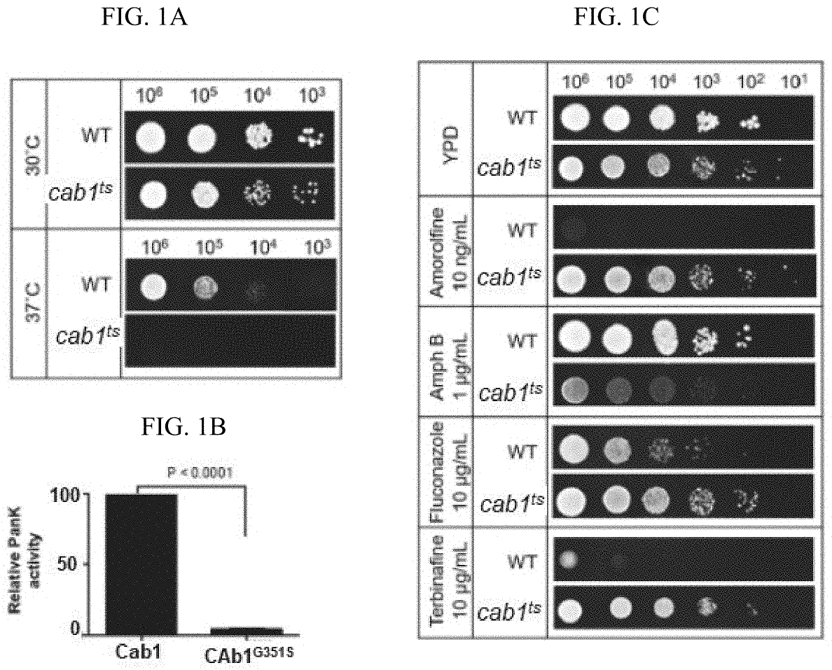

Yeast cells altered in the uptake of pantothenic acid through the Fen2p transporter display reduced susceptibility to fenpropimorph, a morpholine-type antifungal, which targets the enzymes sterol C14-reductase (ERG24) and sterol C8-isomerase (ERG2) in the ergosterol biosynthesis pathway. It was thus investigated whether pantothenic acid utilization regulates ergosterol biosynthesis and yeast sensitivity to antifungals. The first step in pantothenic acid utilization is catalyzed by the pantothenate kinase Cab1p. In yeast, this step is essential for cell viability as knockout of the CAB1 gene results in cell death. Substitution of glycine 351 to serine in CAB1 results in the inability of yeast cells to grow at 37° C. and is thus named the cab lts strain for its thermosensitivity 8,9 (FIG. 1A). Biochemical analyses showed that the activity of the mutant cab1G351S pantothenate kinase is only −7% that of the wild type at 30° C. (FIG. 1B). The availability of the yeast cab1^{ts} mutant made it possible to assess the effect of the altered pantothenate kinase activity on yeast susceptibility to antifungals at 30° C. As shown in FIG. 1C, whereas the growth of WT cells was completely blocked in the presence of the morpholine-type drug amorolfine (10 ng/mL) or was greatly reduced in the presence of fluconazole (10 μg/mL) and terbinafine (10 μg/mL), the growth of cab1^{ts} cells was not affected by these drugs (FIG. 1C). Conversely, with a sub-lethal dose of amphotericin B (1 μg/mL), the growth of WT was only slightly inhibited whereas that of the cab1^{ts} was reduced dramatically in the presence of the compound (FIG. 1C).

### Example 2: Modulation of Cab1p Activity Affects Terbinafine and Amphotericin B Susceptibility

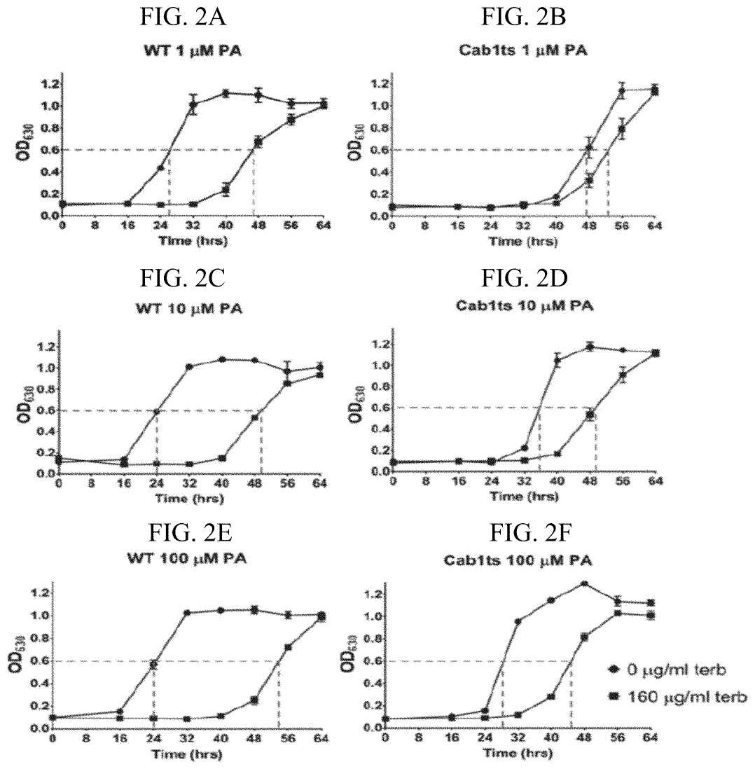

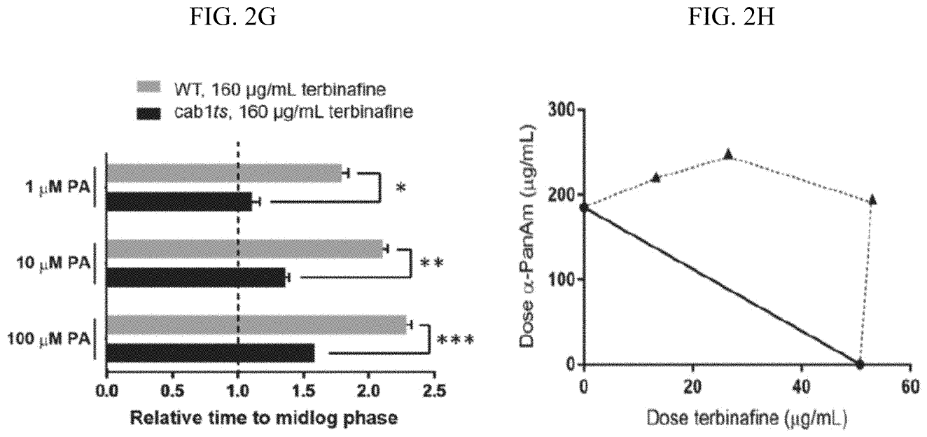

To further investigate the link between pantothenate utilization and antifungal susceptibility, analysis on the effect of inhibition of Cab1p activity and susceptibility to terbinafine and amphotericin B was performed. The growth of cab1ts cells at 30° C. can be significantly improved in media supplemented with increased concentrations of pantothenic acid, as substrate availability compensates for the weak activity of the mutant enzyme. The growth rates of WT and cab1ts cells were measured in minimal medium supplemented with 1, 10 or 100 μM pantothenic acid in the absence or presence of terbinafine. As shown in FIG. 2, the growth of the cab1^{t}s mutant was ameliorated with pantothenic acid supplementation as the time to mid-log phase decreased from 48 hours to 36 hours and 28 hours by increasing the concentration of exogenous pantothenic acid from 1 μM to 10 μM and 100 μM, respectively (FIGS. 2B, 2D, 2F). Comparatively, the growth of WT cells was not significantly altered by the increasing concentrations of pantothenic acid, as the time to mid-log phase was ˜26, 24, and 24 hours at 1 μM, 10 μM, and 100 μM PA, respectively (FIGS. 2A, 2C, 2E). Consistent with the results seen on YPD agar, the growth of WT cells was dramatically decreased in liquid media supplemented with 1 μM PA in the presence of terbinafine (relative time to mid-log phase of 1.8 in the presence vs. absence of the drug) whereas that of the cab1^{t}s mutant was only moderately affected by the drug (relative time to mid-log phase of 1.1 in the presence vs. absence of the drug) (FIG. 2G). Addition of pantothenic acid to the culture medium at 10 μM and 100 μM resulted in higher sensitivity of both WT cells (relative time to mid-log phase of 2.1 and 2.3 in the presence of 10 and 100 μM, respectively) and cab1^{ts} cells (relative time to mid-log phase of 1.4 and 1.6 in the presence of 10 and 100 μM, respectively) to the drug (FIG. 2G).

Because reduced activity of Cab1p in the cab1ts cells correlated with reduced susceptibility to terbinafine, it was reasoned that chemical inhibition of Cab1p activity and/or CoA biosynthesis in the WT background could also result in reduced susceptibility to the drug. The susceptibility of WT cells to terbinafine in the presence of the pantothenamide α-PanAm, which is both a substrate of the kinase Cab1p phosphorylation and a competitive inhibitor of that enzyme and inhibits pantothenate utilization, was examined. As shown in the isobologram in FIG. 2H, terbinafine and α-PanAm displayed a typical drug-drug antagonism pattern in WT cells, demonstrating that inhibition of Cab1p activity and pantothenate utilization results in reduced susceptibility to terbinafine.

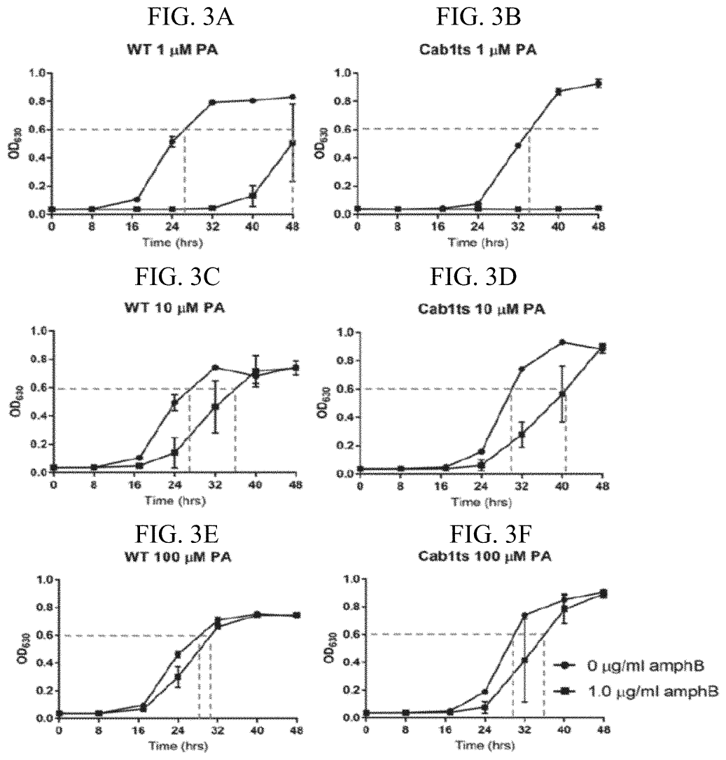

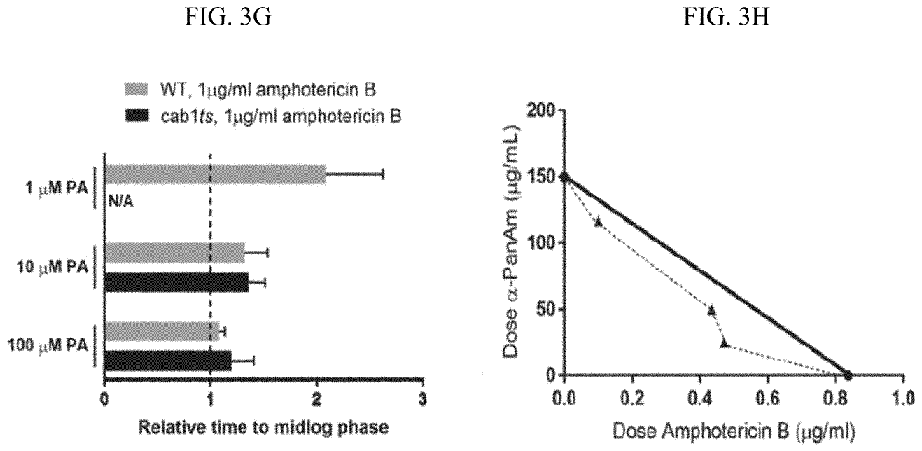

Similar investigations were carried out for WT and cab1^{ts} cells in the presence of varying PA concentrations for amphotericin B. Again, it was demonstrated that supplementation of PA did not significantly affect WT growth patterns, as the time to mid-log phase was ˜26, 26, and 28 hours at 1 μM, 10 μM, and 100 μM PA, respectively (FIGS. 3A, 3C, 3E), while cab1^{ts} cells again demonstrated improved growth with higher exogenous PA concentrations, with time to mid-log phases ˜34, 30, and 29 hours, respectively (FIGS. 3B, 3D, 3F). Consistent with the effect of amphotericin B on YPD agar, the growth of WT cells was not significantly affected at 100 μM PA in the presence of 1 μg/mL amphotericin B, but was increasingly and significantly affected as the exogenous PA concentration was lowered to 1 μM (relative time to mid-log phase of 2.1 in the presence vs. absence of the drug) (FIG. 3G). The cab1^{ts} cells showed a similar trend as the WT cells, displaying more sensitivity to amphotericin B as exogenous PA was reduced. However, it is apparent that the mutant strain was more sensitive to the drug, especially noting that the strain did not grow whatsoever in the presence of the drug when only 1 μM PA medium was used (FIG. 3G). Analysis of the combined effect of amphotericin B and α-PanAm showed a typical drug-drug synergism pattern in WT cells, demonstrating that inhibition of Cab1p activity results in increased susceptibility to amphotericin B (FIG. 3H).

### Example 3: Inhibition of Cab1p Activity Results in Reduced Squalene and Lanosterol Levels

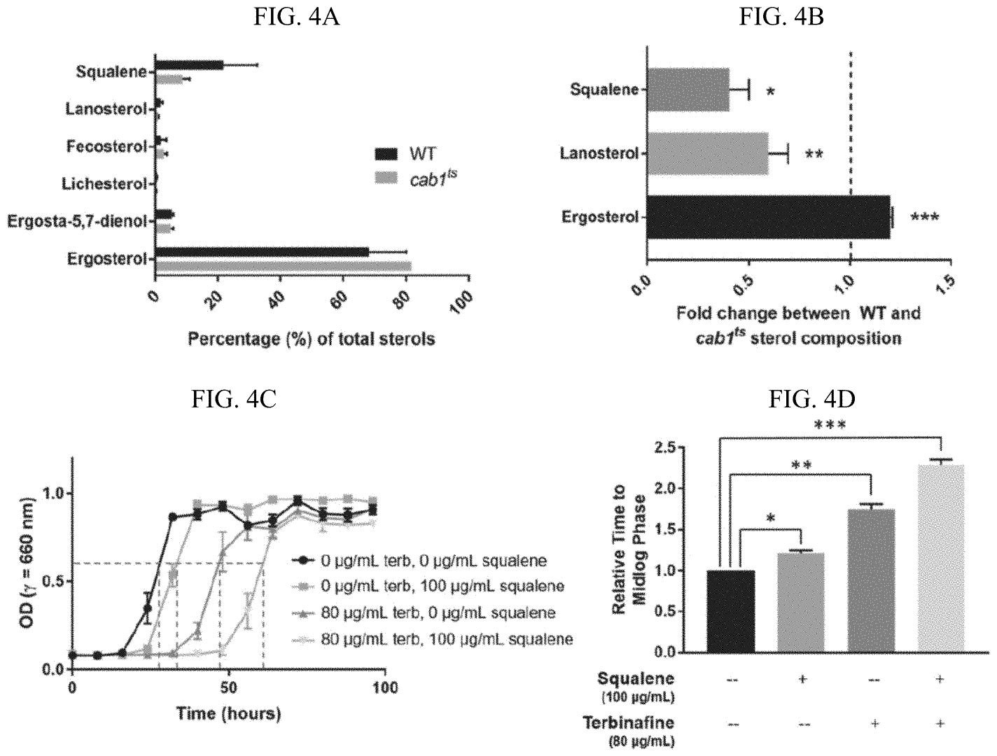

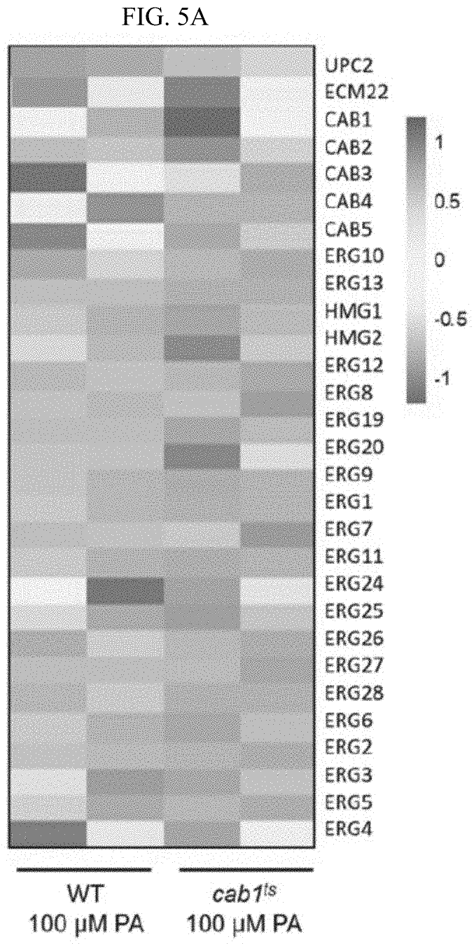

To investigate the mechanism by which modulation of Cab1p activity controls susceptibility to antifungals, the sterol composition of WT and cab1^{ts} strains was examined under permissive conditions. Squalene, steroidal ergosterol precursors and ergosterol were analyzed by GC-MS. Ergosterol comprised the largest percentage of total sterols in both WT and cab1^{ts} cells, followed by the open-chain sterol precursor squalene. All other sterols each comprised no more than ˜10% of total sterols (FIG. 4A). As shown in FIG. 4B, reduced pantothenate phosphorylation in the cab1^{ts} mutant resulted in a significant reduction in the levels of squalene (p-value 0.0088) and lanosterol (p-value 0.0185), and a significant increase in ergosterol content (p-value 0.0009). Other sterols were not analyzed further due to their trace amounts in both WT and cab1ts cells. Consistent with these findings, supplementation of the growth medium of WT cells with 100 μg/mL of squalene had only a slight effect on cell growth (mid-log phase reached at 28 hours in the absence of squalene vs. 34 hours in the presence of squalene), whereas in the presence of terbinafine, the growth of WT cells was dramatically reduced (mid-log phase reached at 47 hours in the absence of squalene vs. 61 hours in the presence of squalene) (FIG. 4C, 4D).

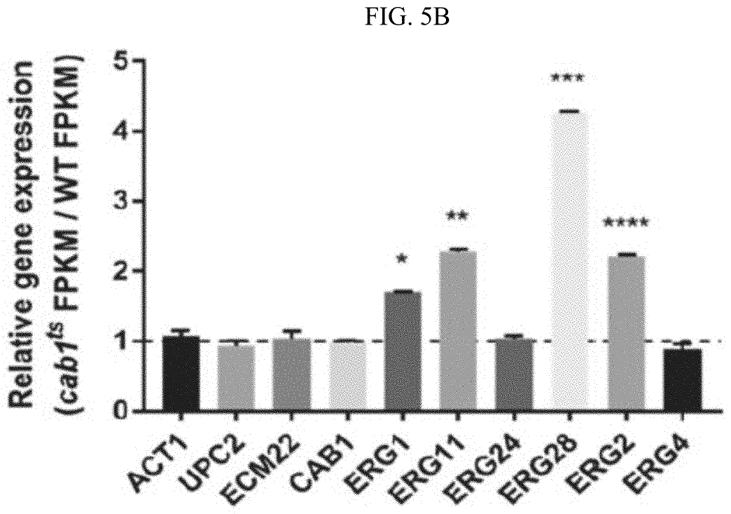

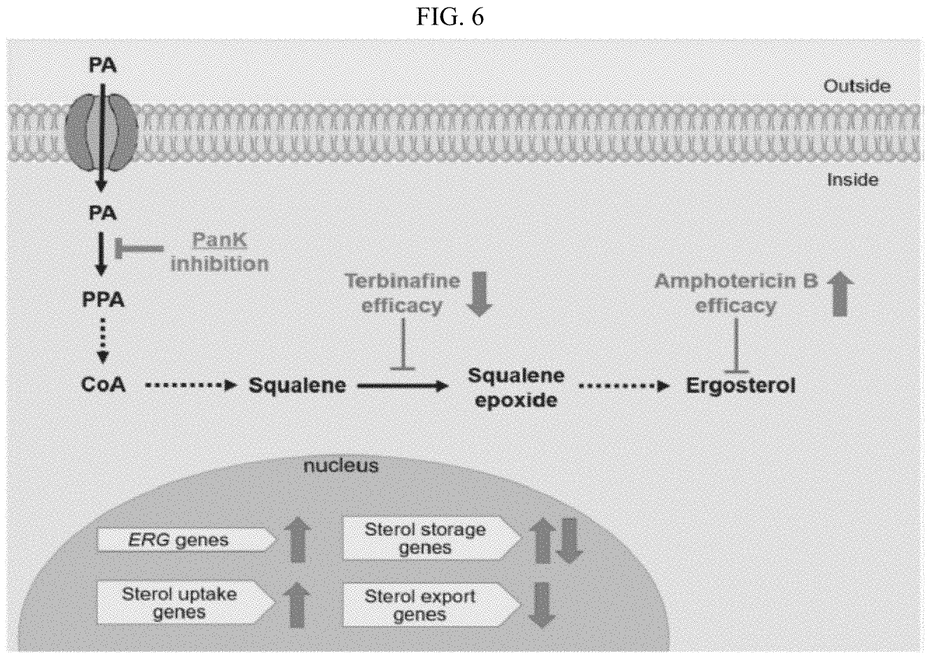

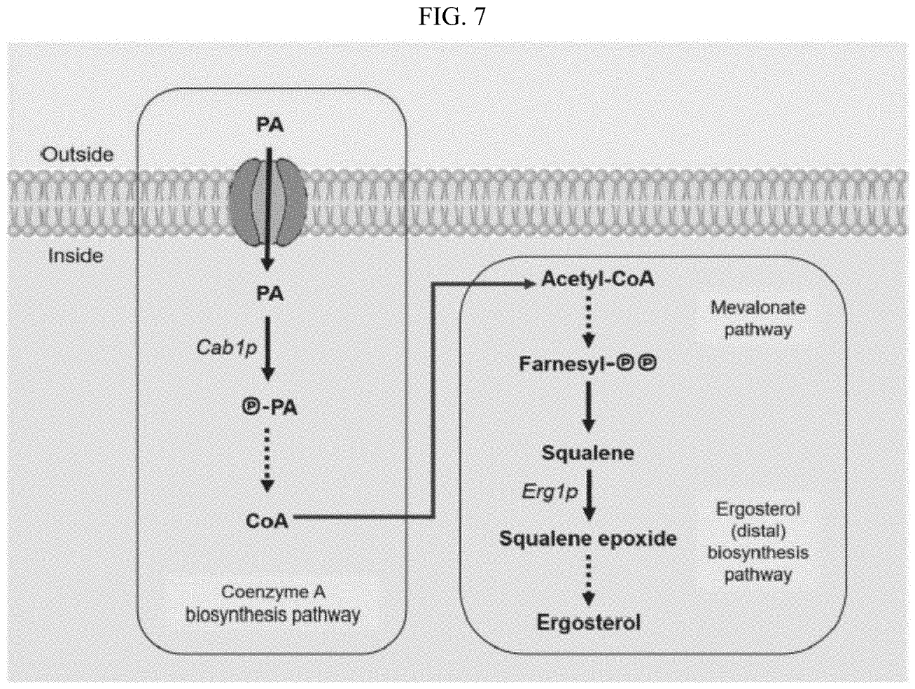

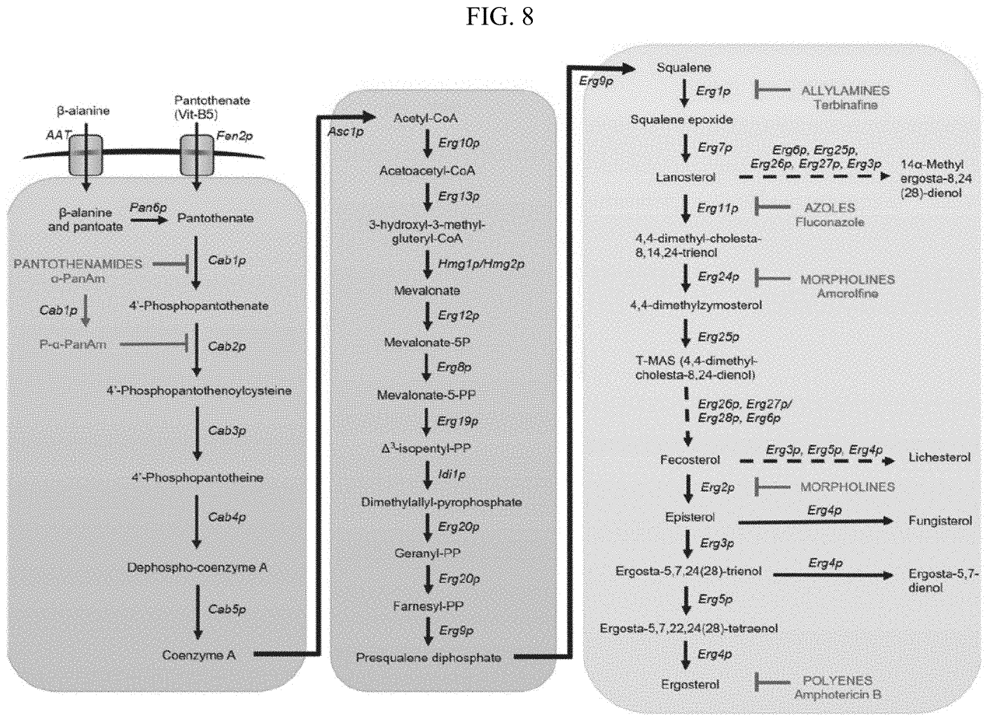

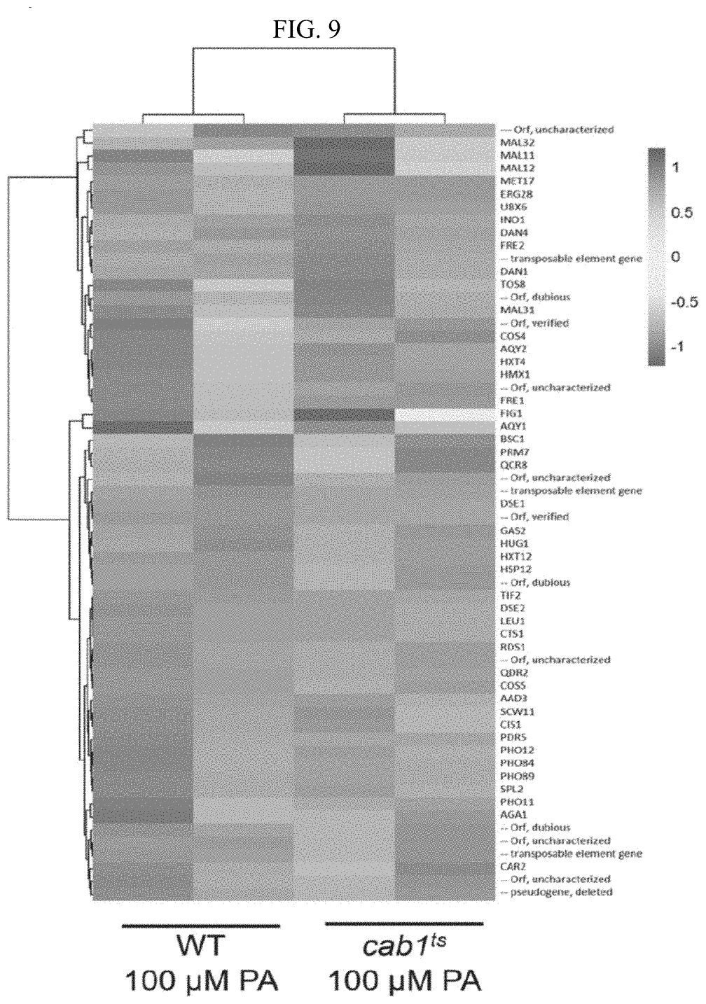

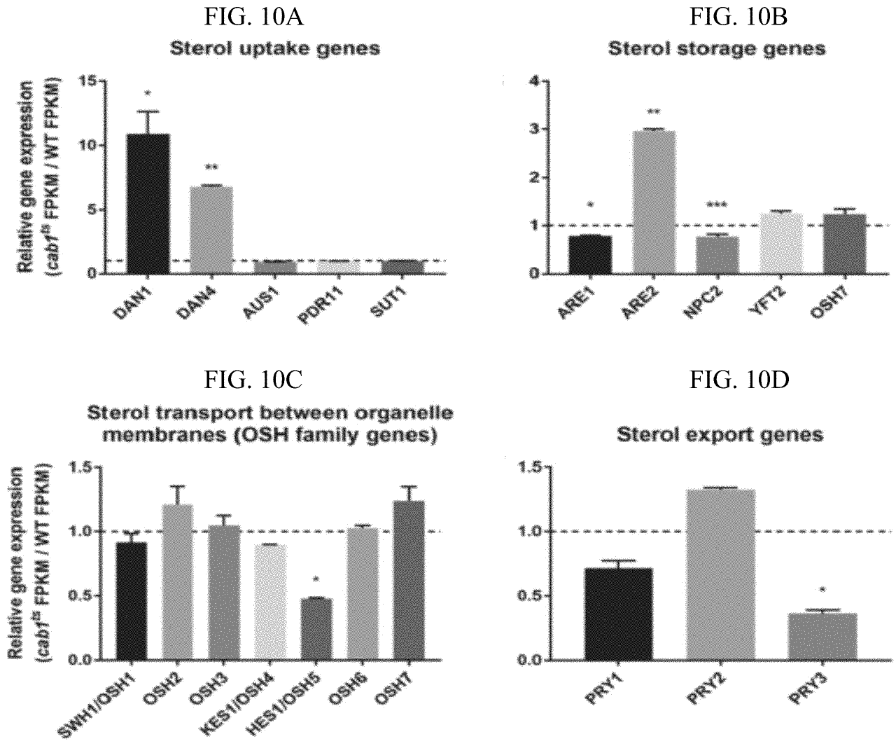

To determine whether the reduced level of squalene seen in the cab1^{ts} mutant could also be due to changes in the transcription of the ERG1 gene or other genes in the ERG biosynthetic pathway, RNAseq analysis was performed on WT and cab1^{ts} strains grown under permissive conditions (minimal medium in the presence of 100 μM pantothenic acid only). Two high-quality mRNA samples were selected for RNAseq analysis from two independent replicates and the expression of the CoA biosynthesis genes (CAB1-5), ergosterol biosynthesis genes (20 ERG genes) and the transcriptional factors UPC2 and ECM22 were examined (FIG. 5A). Of the 20 ERG genes analyzed, the expression of ERG1, ERG11, ERG28, and ERG2 was 1.8-fold (p-value 0.0011), 2.5-fold (p-value 0.0013), 4.6-fold (p-value 0.0003), and 2.4-fold (p-value 0.0004) induced, respectively, in the cab1^{ts} mutant compared to WT (FIG. 5B). The ERG4 gene, a sterol reductase catalyzing the final step in ergosterol biosynthesis, was the only gene in the ERG biosynthesis pathway found to be downregulated in the mutant, though not significantly. The expression of the transcriptional factors UPC2 and ECM22 was not significantly different between the WT and the cab1ts mutant (FIG. 5B). As a control, no significant differences between the WT and cab1ts strains could be detected in the expression levels of the housekeeping ACT1 gene (FIG. 5B).

### Example 4

Efforts were made to identify inhibitors of pantothenate kinase (PanK), which plays an essential role in fungal viability. Initially, cloning and expression of PanKs from different fungal pathogens (Candida albicans, Candida auris, Aspergillus fumigatu and Histoplasma capsulatum) were implemented. Screening of compounds against the A. fumigatus enzyme (AfPanK) allowed for the identification of various active compounds, which are exemplified in Table 1.

In Table 1, “mixed” indicates a compound that is an activator at lower concentrations and is an inhibitor at higher concentrations. % growth inhibition S. cerevisiae monster was determined at 200 μM of compound in VFM medium with no pantothenic acid at 24 h (10⁵ cells/ml).

TABLE 1

| | | | % growth | |
|---|---|---|---|---|
| | | | inhibition | |
| | | | S. cerevisiae | |
| | | | monster at | EC₅₀ |
| | A. fum. | hPanK3 | 200 μM | HeLa Tox |
| Compound | IC₅₀ μM | IC₅₀ μM | (μg/mL) | 72 hr μM |
| A1 | 0.036 | Mixed | 98% (88 μg/mL) | Inactive |
| A2 | 0.083 | Mixed | 87% (99 μg/mL) | Inactive |
| A3 | 0.086 | >5 | 87% (68 μg/mL) | >100 |
| A4 | 2.4 | >100 | 62% (79 μg/mL) | |
| A5 | 5.5 | >100 | 86% (76 μg/mL) | Inactive |
| | | Activator | | |
| A6 | 31 | >100 | 34% (82 μg/mL) | >100 |
| | | Activator | | |
| A7 | >50 | >100 | 79% (83 μg/mL) | |
| A8 | >50 | >100 | 67% (82 μg/mL) | |
| A9 | >50 | >100 | 102% (76 μg/mL) | |
| A10 | 53 | | 13% (71 μg/mL) | |
| A11 | 62 | | 77% (91 μg/mL) | |
| A12 | 68 | | 94% (88 μg/mL) | |
| A13 | 71 | Mixed | 25% (79 μg/mL) | >100 |
| A14 | 78 | | 49% (91 μg/mL) | |
| A15 | 98 | | 37% (85 μg/mL) | |
| A16 | ~100 | >100 | 80% (83 μg/mL) | 2.3 |
| A17 | ~100 | >100 | 63% (86 μg/mL) | |
| A18 | ~100 | >100 | 29% (79 μg/mL) | |

### Example 5

Compounds of interest were assayed for their activity as inhibitors of fungal pantothenate kinase (PanK), modulators of human PanK, and general toxicity against HeLa cells. All compounds in Table 2 were found to be activators of human PanK

TABLE 2

| | | A cer. | | EC₅₀ |
|---|---|---|---|---|
| | A. fum. | Cab1 | hPanK3 | HeLa Tox |
| Compound | IC₅₀ μM | IC₅₀ μM | AC₅₀ μM | 72 hr μM |
| Compound B1 | 0.25 | 0.43 | 0.0017 | Inactive |
| Compound B2 | 0.27 | >100 | 0.0053 | >100 |
| Compound B3 | 10 | >100 | 0.024 | Inactive |
| Compound B4 | 11 | >100 | 0.032 | Inactive |
| Compound B5 | 28 | 9.4 | 0.038 | >100 |
| Compound B6 | 0.26 | 0.19 | 0.058 | Inactive |
| Compound B7 | 44 | >100 | 0.076 | >100 |
| Compound B8 | 0.89 | 0.55 | 0.16 | Inactive |
| Compound B9 | 1.8 | 30 | 0.27 | >100 |

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A pharmaceutical composition comprising at least one pharmaceutically acceptable carrier and at least one compound of formula (Ia) or (Ib), or a salt, solvate, isotopically labelled derivative, stereoisomer, tautomer, or geometric isomer thereof, and any mixtures thereof: 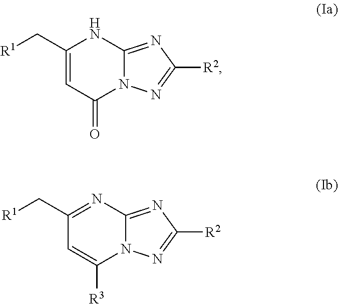
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃— (optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃-(optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl;
with the proviso that the compound is not:

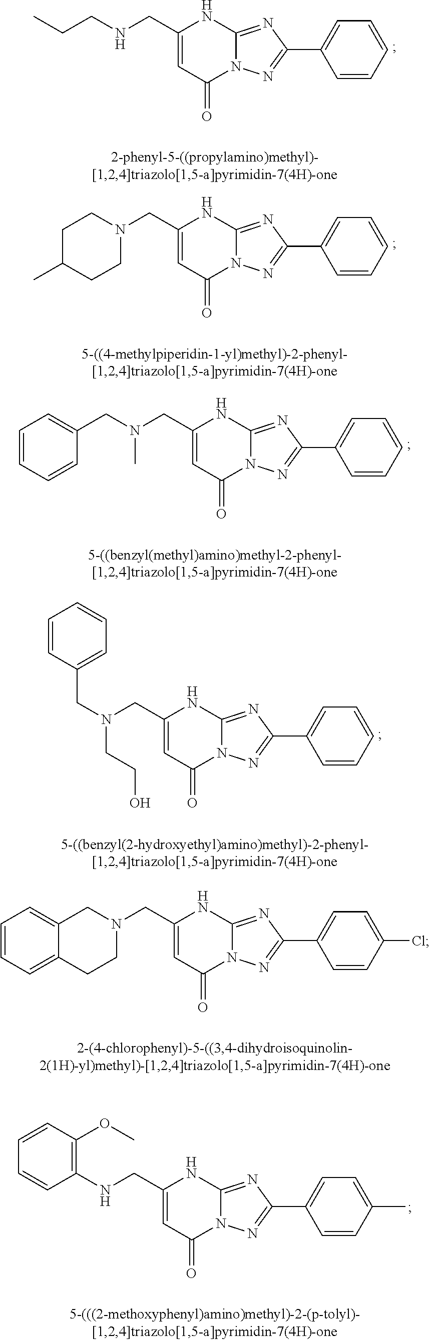 [SMILES: CCCNCc1cc(=O)n2nc(-c3ccccc3)nc2[nH]1 ; CC1CCN(Cc2cc(=O)n3nc(-c4ccccc4)nc3[nH]2)CC1 ; CN(Cc1ccccc1)Cc1cc(=O)n2nc(-c3ccccc3)nc2[nH]1 ; O=c1cc(CN(CCO)Cc2ccccc2)[nH]c2nc(-c3ccccc3)nn12 ; O=c1cc(CN2CCc3ccccc3C2)[nH]c2nc(-c3ccc(Cl)cc3)nn12 ; COc1ccccc1NCc1cc(=O)n2nc(-c3ccc(C)cc3)nc2[nH]1]

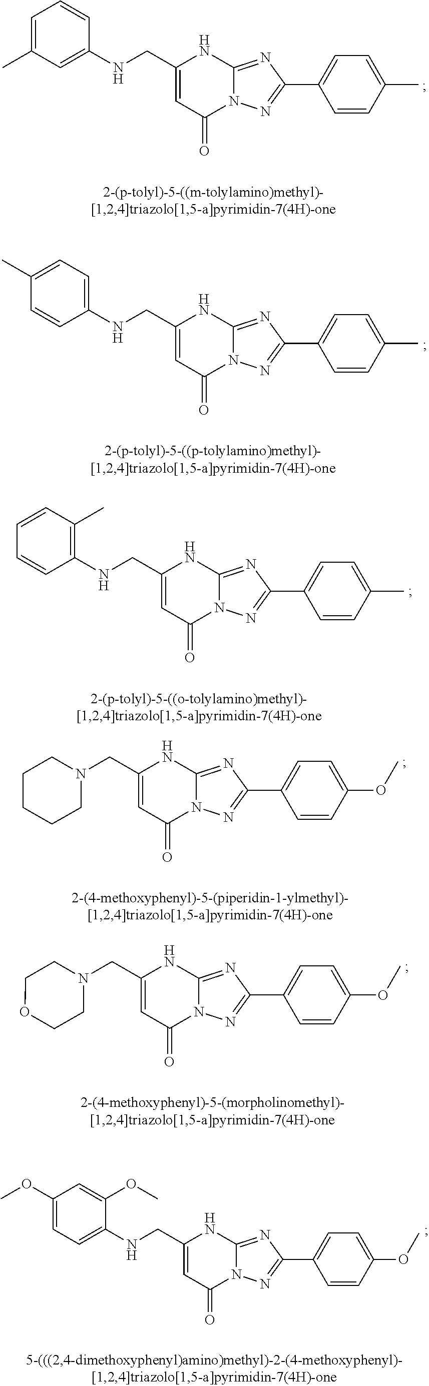

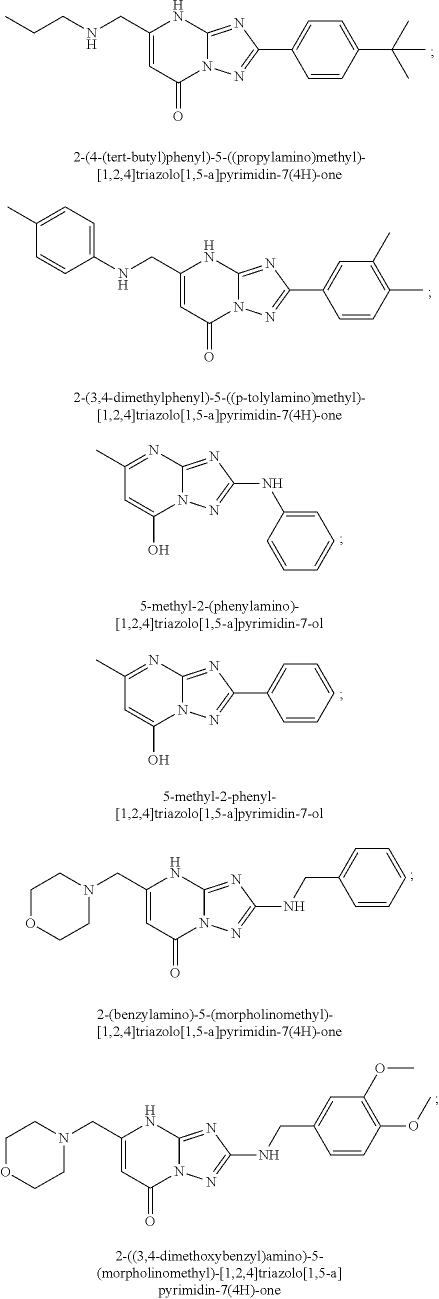

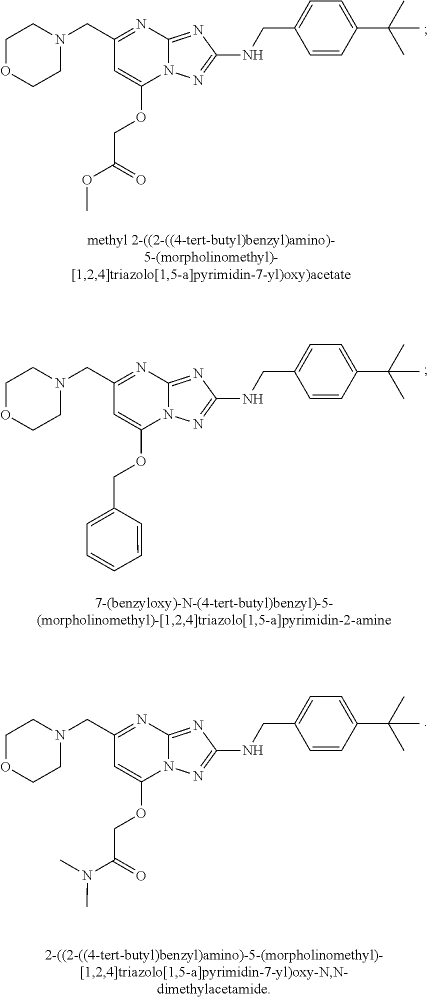 [SMILES: COC(=O)COc1cc(CN2CCOCC2)nc2nc(NCc3ccc(C(C)(C)C)cc3)nn12 ; CC(C)(C)c1ccc(CNc2nc3nc(CN4CCOCC4)cc(OCc4ccccc4)n3n2)cc1 ; CN(C)C(=O)COc1cc(CN2CCOCC2)nc2nc(NCc3ccc(C(C)(C)C)cc3)nn12]

2. The pharmaceutical composition of claim 1, wherein R′ is selected from the group consisting of H, —NR^{4a}R^{4b}, NR^{4c} (optionally substituted phenyl), and optionally substituted N-linked heterocyclyl.

3. (canceled)

4. The pharmaceutical composition of claim 1, wherein R³ is —NR^{4d}R^{4e} or —C(═O)OR^{4d}

5. The pharmaceutical composition of claim 1, wherein each phenyl in R² is independently substituted with at least one C₂-C₈ alkyl, optionally wherein each phenyl in R² is independently substituted at the para-position with at least one C₂-C₈ alkyl.

6. (canceled)

7. The pharmaceutical composition of claim 1, wherein at least one of the following applies:
(a) the N-linked heterocyclyl comprises aziridinyl, azetidinyl, pyrrolidinyl, pyrazolidinyl, piperidinyl, 1,2,3,6-tetrahydropyridinyl, 1,4-dihydropyridinyl, piperazinyl, morpholinyl, thiomorpholinyl, homopiperazinyl, or homopiperidinyl;
(b) the heteroaryl comprises imidazolyl, pyridinyl, pyrimidinyl, pyrazinyl, thienyl, furyl, pyrrolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, or 1,3,4-oxadiazolyl.

8. (canceled)

9. The pharmaceutical composition of claim 1, wherein the compound is at least one of:

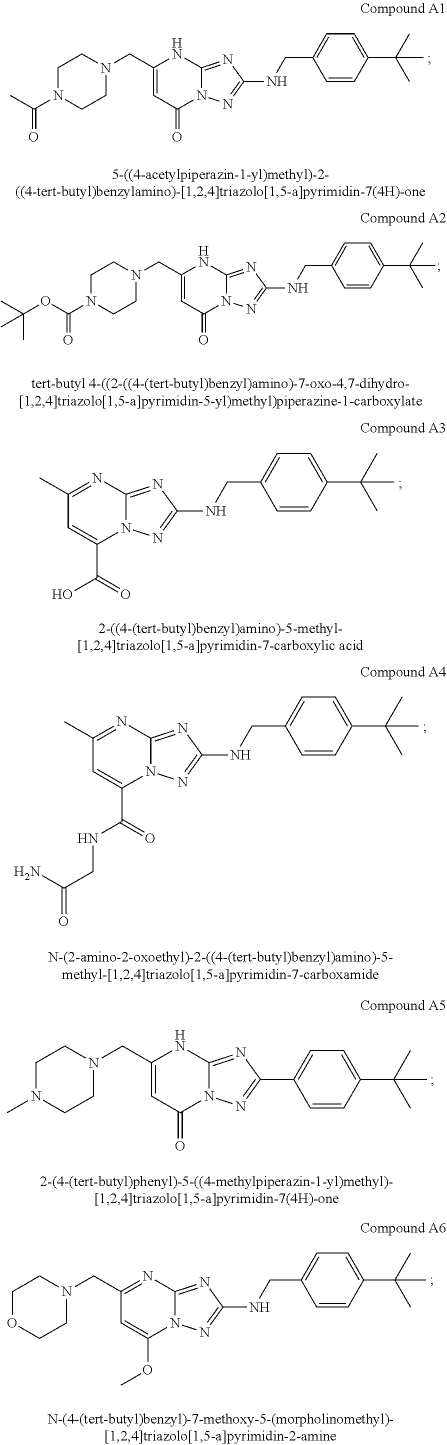

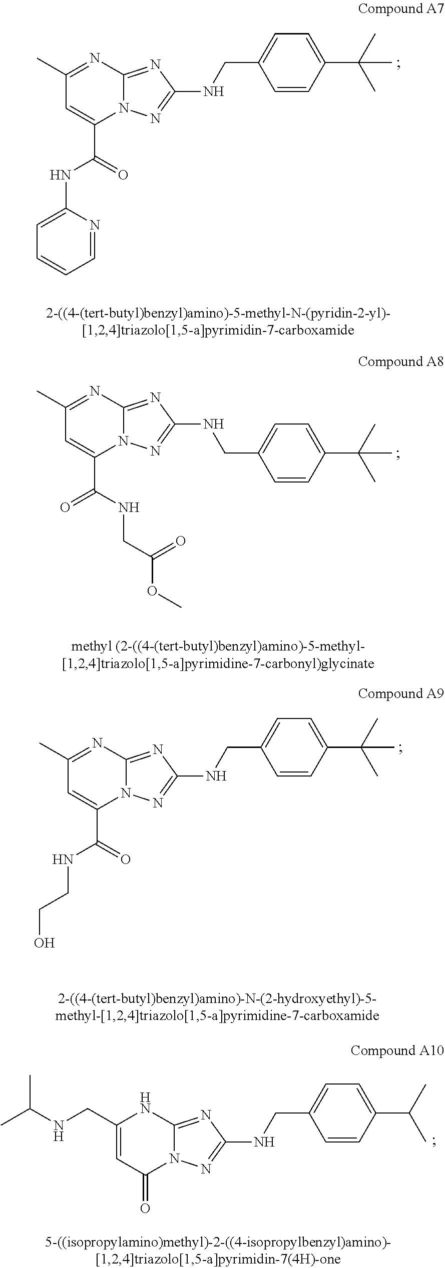

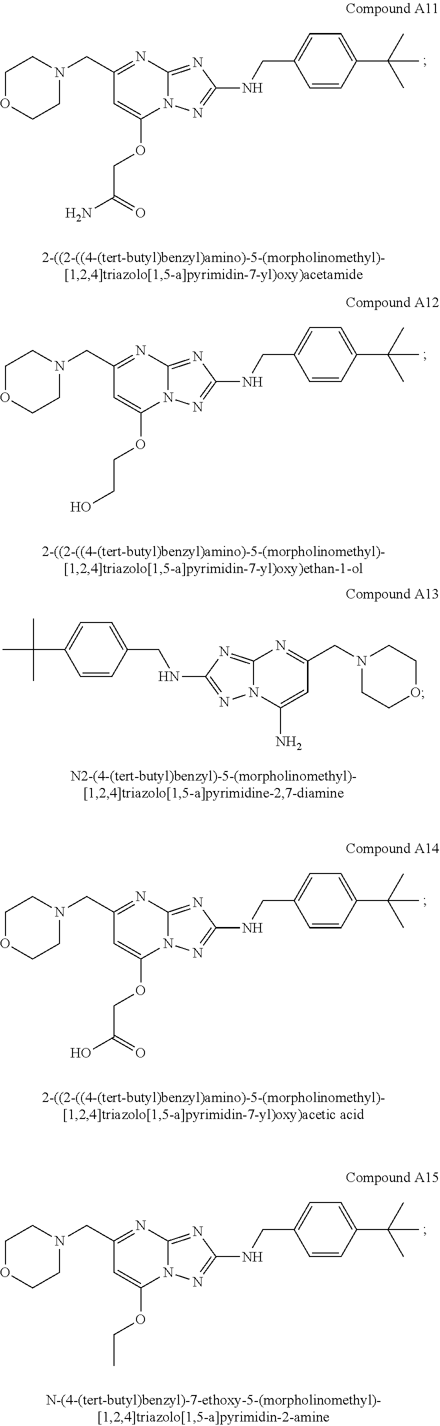

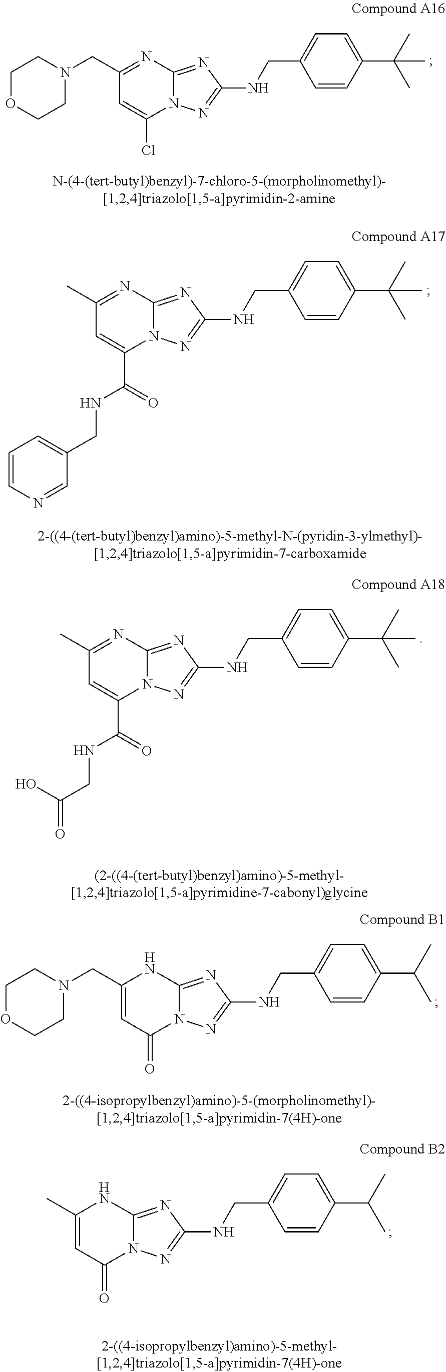

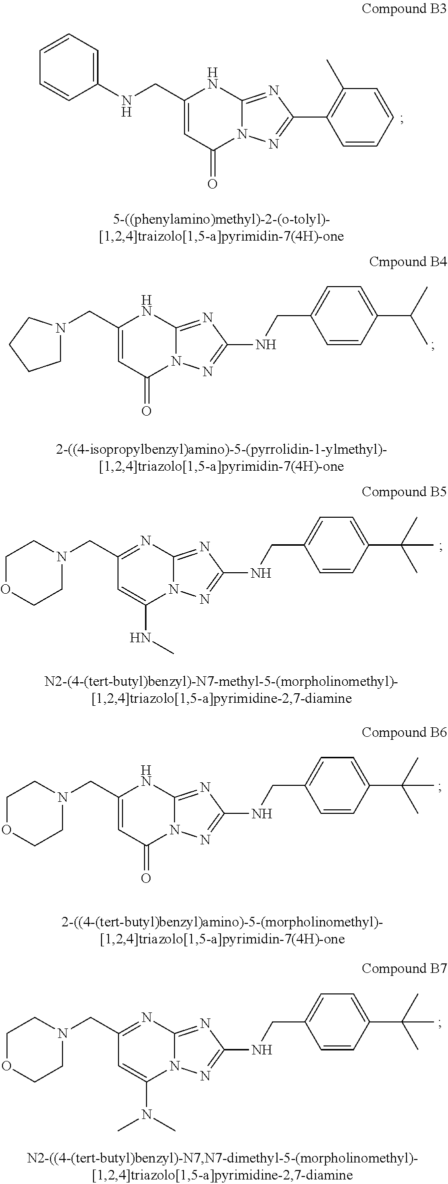 [SMILES: Cc1ccccc1-c1nc2[nH]c(CNc3ccccc3)cc(=O)n2n1 ; CC(C)c1ccc(CNc2nc3[nH]c(CN4CCCC4)cc(=O)n3n2)cc1 ; CNc1cc(CN2CCOCC2)nc2nc(NCc3ccc(C(C)(C)C)cc3)nn12 ; CC(C)(C)c1ccc(CNc2nc3[nH]c(CN4CCOCC4)cc(=O)n3n2)cc1 ; CN(C)c1cc(CN2CCOCC2)nc2nc(NCc3ccc(C(C)(C)C)cc3)nn12]

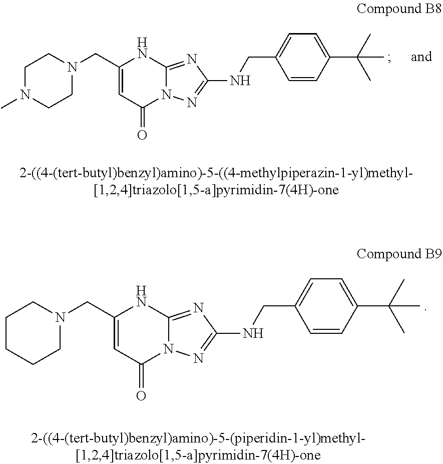 [SMILES: CN1CCN(Cc2cc(=O)n3nc(NCc4ccc(C(C)(C)C)cc4)nc3[nH]2)CC1 ; CC(C)(C)c1ccc(CNc2nc3[nH]c(CN4CCCCC4)cc(=O)n3n2)cc1]

10. The pharmaceutical composition of claim 1, which further comprises an agent useful for treating, ameliorating, and/or preventing a fungal infection in a mammal,
optionally wherein at least one of the following applies:
(a) the at least one additional agent comprises amphotericin B;
(b) the pharmaceutical composition further comprises an agent useful for treating, ameliorating, or preventing a neurological disease or disorder or an Acyl-CoA dehydrogenation deficiency in a mammal.

11. (canceled)

12. (canceled)

13. A compound of formula (Ia) or (Ib), or a salt, solvate, isotopically labelled derivative, stereoisomer, tautomer, or geometric isomer thereof, and any mixtures thereof: 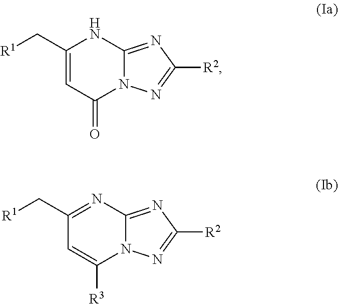
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃-(optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃— (optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR′, optionally substituted phenyl, optionally substituted heteroaryl, optionally substituted heterocyclyl, —C(═O)O^{a}, —OC(═O)R^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl;
with the proviso that the compound is not:

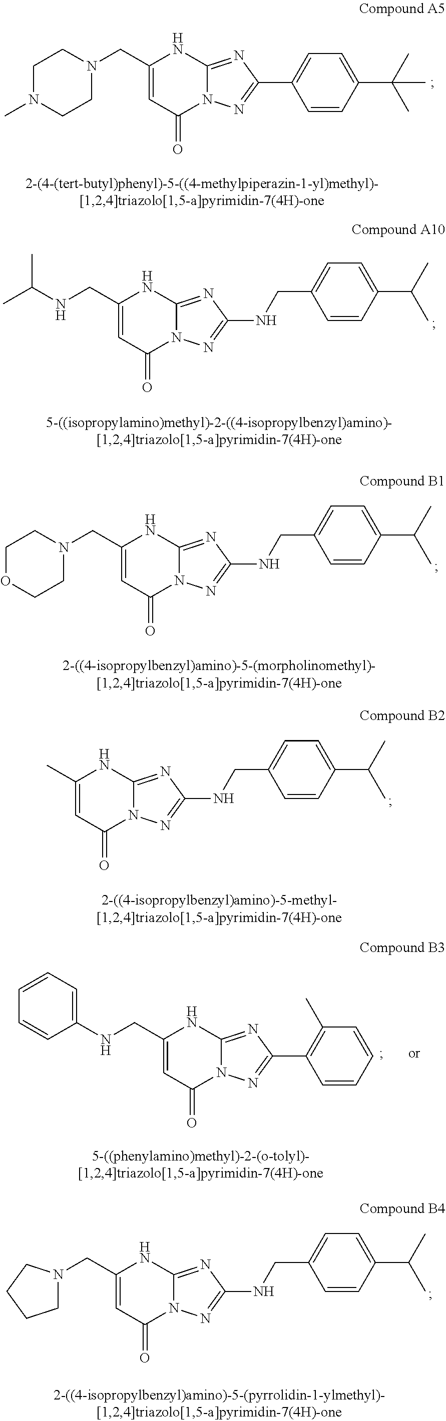

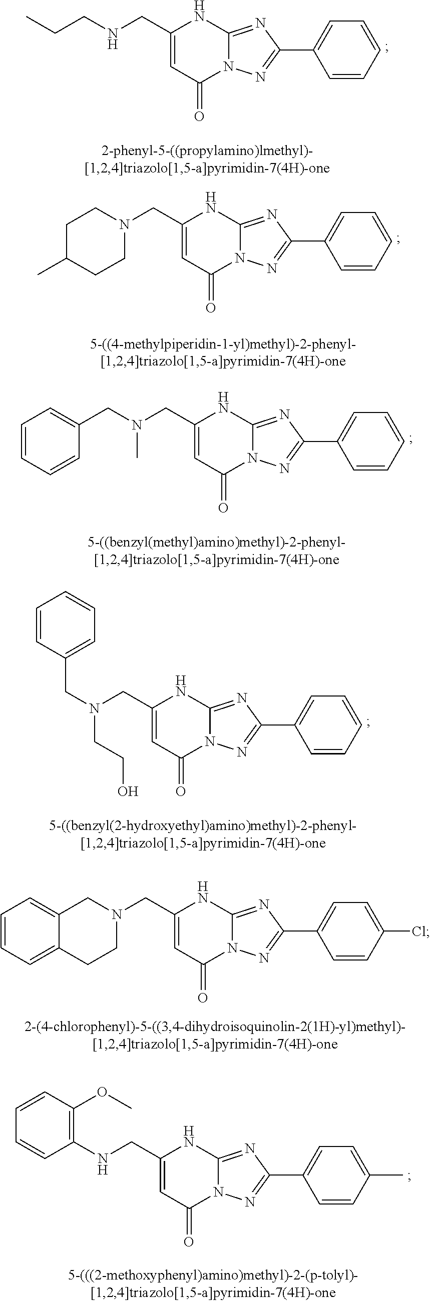 [SMILES: CCCNCc1cc(=O)n2nc(-c3ccccc3)nc2[nH]1 ; CC1CCN(Cc2cc(=O)n3nc(-c4ccccc4)nc3[nH]2)CC1 ; CN(Cc1ccccc1)Cc1cc(=O)n2nc(-c3ccccc3)nc2[nH]1 ; O=c1cc(CN(CCO)Cc2ccccc2)[nH]c2nc(-c3ccccc3)nn12 ; O=c1cc(CN2CCc3ccccc3C2)[nH]c2nc(-c3ccc(Cl)cc3)nn12 ; COc1ccccc1NCc1cc(=O)n2nc(-c3ccc(C)cc3)nc2[nH]1]

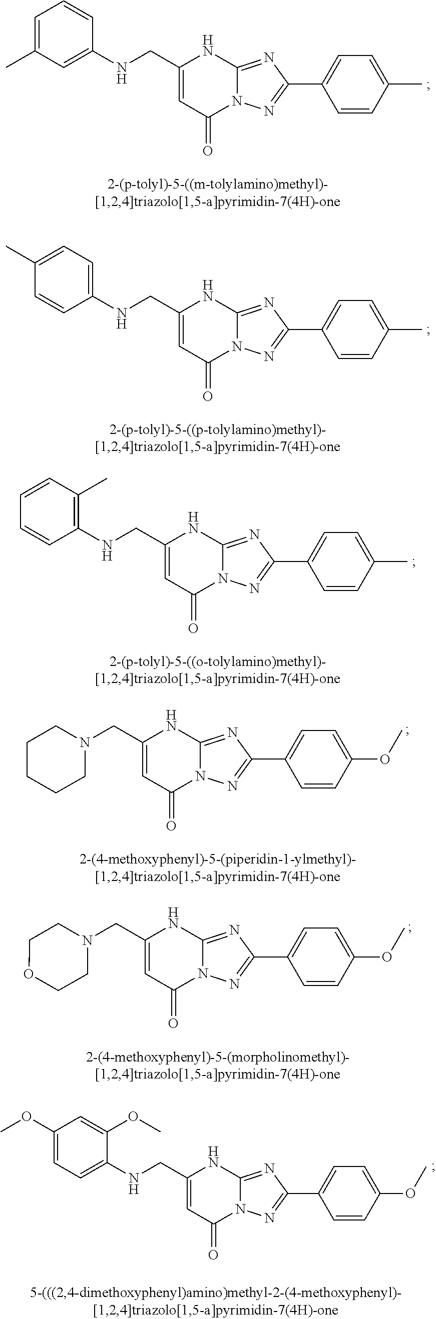

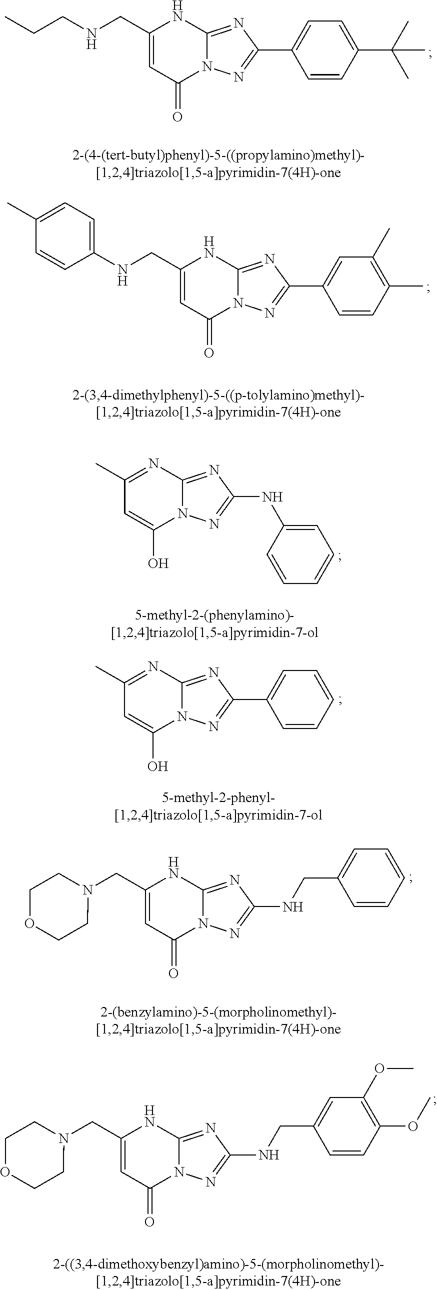

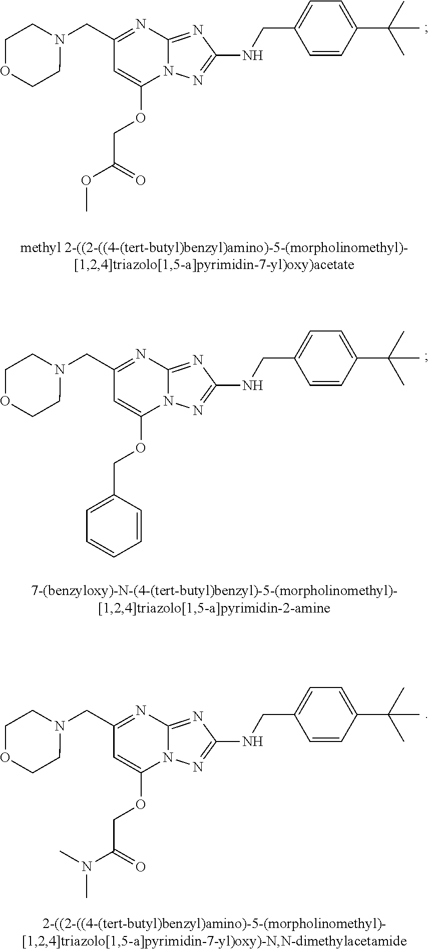 [SMILES: COC(=O)COc1cc(CN2CCOCC2)nc2nc(NCc3ccc(C(C)(C)C)cc3)nn12 ; CC(C)(C)c1ccc(CNc2nc3nc(CN4CCOCC4)cc(OCc4ccccc4)n3n2)cc1 ; CN(C)C(=O)COc1cc(CN2CCOCC2)nc2nc(NCc3ccc(C(C)(C)C)cc3)nn12]

14. The compound of claim 13, wherein R¹ is selected from the group consisting of H, —NR^{4a}R^{4b}, NR^{4c} (optionally substituted phenyl), and optionally substituted N-linked heterocyclyl.

15. (canceled)

16. The compound of claim 13, wherein R³ is —NR^{4d}R^{4e} or —C(═O)OR^{4d}.

17. The compound of claim 13, wherein each phenyl in R² is independently substituted with at least one C₂-C₈ alkyl, optionally wherein each phenyl in R² is independently substituted at the para-position with at least one C₂-C₈ alkyl.

18. (canceled)

19. The compound of claim 13, wherein at least one of the following applies:
(a) the N-linked heterocyclyl comprises aziridinyl, azetidinyl, pyrrolidinyl, pyrazolidinyl, piperidinyl, 1,2,3,6-tetrahydropyridinyl, 1,4-dihydropyridinyl, piperazinyl, morpholinyl, thiomorpholinyl, homopiperazinyl, or homopiperidinyl;
(b) the heteroaryl comprises imidazolyl, pyridinyl, pyrimidinyl, pyrazinyl, thienyl, furyl, pyrrolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, or 1,3,4-oxadiazolyl.

20. (canceled)

21. The compound of claim 13, which is at least one of:

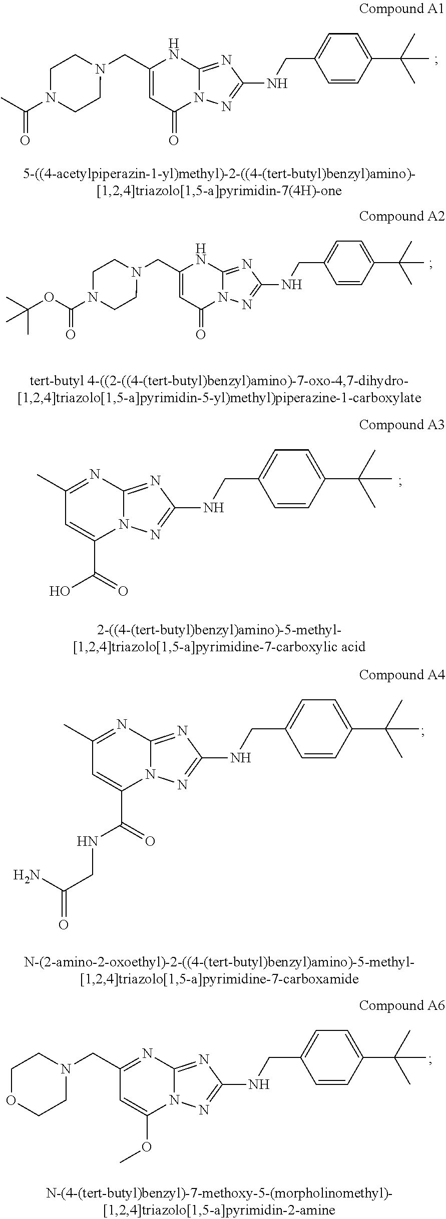

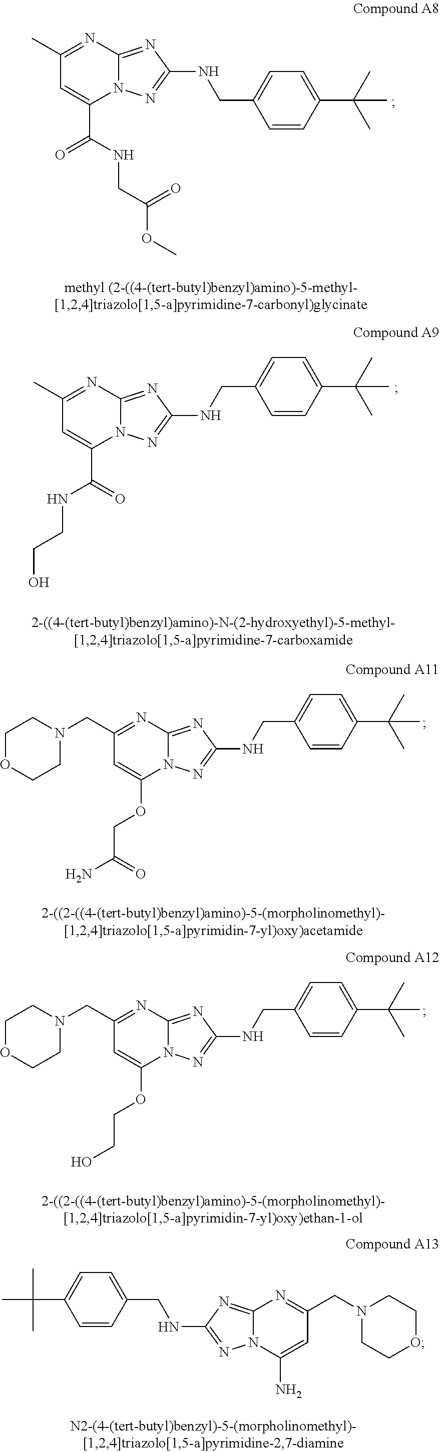

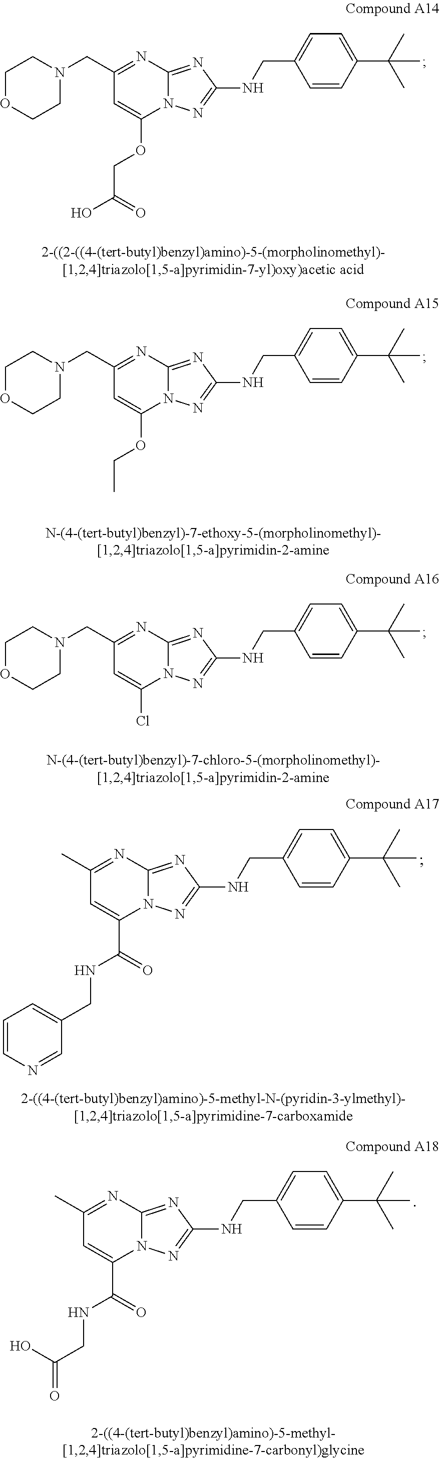 [SMILES: CC(C)(C)c1ccc(CNc2nc3nc(CN4CCOCC4)cc(OCC(=O)O)n3n2)cc1 ; CCOc1cc(CN2CCOCC2)nc2nc(NCc3ccc(C(C)(C)C)cc3)nn12 ; CC(C)(C)c1ccc(CNc2nc3nc(CN4CCOCC4)cc(Cl)n3n2)cc1 ; Cc1cc(C(=O)NCc2cccnc2)n2nc(NCc3ccc(C(C)(C)C)cc3)nc2n1 ; Cc1cc(C(=O)NCC(=O)O)n2nc(NCc3ccc(C(C)(C)C)cc3)nc2n1]

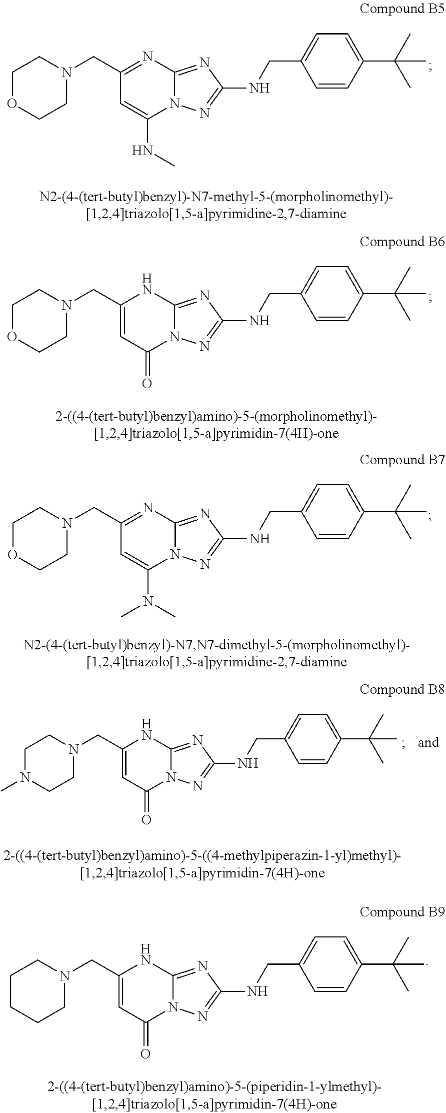

22. A method of inhibiting pantothenate kinase (PanK) in a fungus, wherein the method comprises contacting the fungus with a compound of formula (Ia) or (Ib), or a salt, solvate, isotopically labelled derivative, stereoisomer, tautomer, or geometric isomer thereof, and any mixtures thereof: 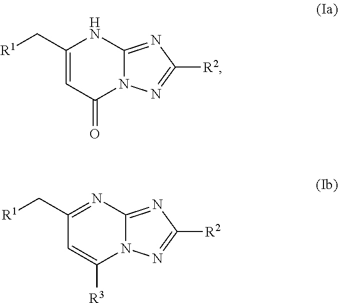
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃-(optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃-(optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR^{a}, optionally substituted phenyl, optionally substituted heteroaryl, optionally substituted heterocyclyl, —C(═O)OR^{a}, —OC(═O)R^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl;
optionally wherein the fungus is on the skin of a mammalian subject or within a mammalian subject.

23. (canceled)

24. A method of treating, ameliorating, and/or preventing a fungal infection in a subject, wherein the method comprises administering to the subject a therapeutically effective amount of a compound of formula (Ia) or (Ib), or a salt, solvate, isotopically labelled derivative, stereoisomer, tautomer, or geometric isomer thereof, and any mixtures thereof: 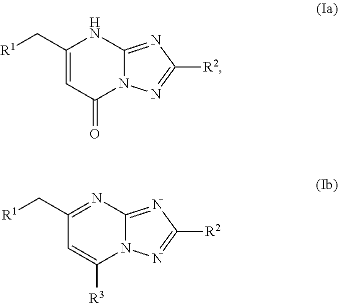
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃— (optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃-(optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR′, optionally substituted phenyl, optionally substituted heteroaryl, optionally substituted heterocyclyl, —C(═O)OR^{a}, —OC(═O)R^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl.

25. The method of claim 24, wherein the fungus comprises at least one of Candida, Aspergillus, Histoplasma, Cryptococcus, and Mucor.

26. The method of claim 24, wherein the compound is the only antifungal agent administered to the subject or wherein the subject is further administered amphotericin B.

27. (canceled)

28. The method of claim 26, wherein at least one of the following applies:
(a) administration of the compound and amphotericin B results in less likely drug resistance occurrence in the subject as compared to an equivalent subject that is administered amphotericin B in the absence of the compound;
(b) administration of the compound and amphotericin B allows for administration of an amount of the amphotericin B that is lower than the corresponding amount of amphotericin B that has to be administered in the absence of the compound to achieve equivalent treatment or prevention of the fungal infection.

29. (canceled)

30. The method of claim 24, wherein the compound inhibits fungal PanK selectively over a human PanK enzyme.

31. The method of claim 24, wherein the subject is a mammal, preferably the mammal is a human.

32. (canceled)

33. A method of activating pantothenate kinase (PanK) in a human cell, wherein the method comprises contacting the human cell with a compound of formula (Ia) or (Ib), or a salt, solvate, isotopically labelled derivative, stereoisomer, tautomer, or geometric isomer thereof, and any mixtures thereof. 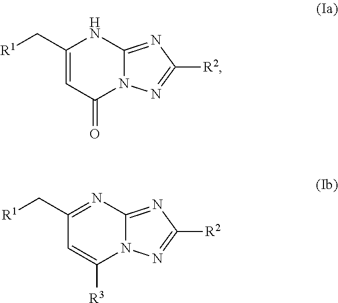
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃— (optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃-(optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR′, optionally substituted phenyl, optionally substituted heteroaryl, optionally substituted heterocyclyl, —C(═O)OR^{a}, —OC(═O)R^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl;
preferably wherein the human cell is ex vivo or in vivo.

34. (canceled)

35. A method of treating, ameliorating, or preventing a disease or disorder associated with reduced or deficient PanK activity, or a disease or disorder associated with reduced cellular free CoA levels, in a human subject, wherein the method comprises administering to the subject a therapeutically effective amount of a compound of formula (Ia) or (Ib), or a salt, solvate, isotopically labelled derivative, stereoisomer, tautomer, or geometric isomer thereof, and any mixtures thereof. 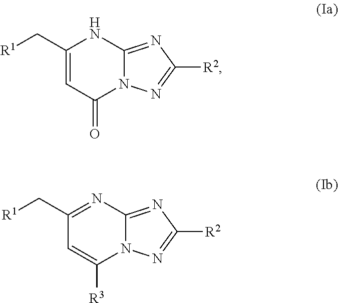
wherein in (Ia) or (Ib):
R¹ is selected from the group consisting of H, —NR^{4a}R^{4b}, —NR^{4c} (optionally substituted phenyl), and optionally substituted N-linked heterocyclyl;
R² is selected from the group consisting of phenyl and —N(R^{4c})—(CH₂)₁₋₃-(optionally substituted phenyl);
R³ is selected from the group consisting of F, Cl, Br, I, —OR^{4d}, —NR^{4d}R^{4e}, —C(═O)OR^{4d}, —C(═O)NR^{4e}R^{4f}, —C(═O)N(R^{4g})—(CH₂)₁₋₃-(optionally substituted phenyl), and —C(═O)N(R^{4h})—(CH₂)₁₋₃-(optionally substituted heteroaryl);
each occurrence of R^{4a}-R^{4h} is independently selected from the group consisting of H, optionally substituted C₁-C₆ alkyl, and optionally substituted C₃-C₈ cycloalkyl;
each occurrence of alkyl, cycloalkyl, phenyl, heterocyclyl, and heteroaryl is optionally independently substituted with at least one of C₁-C₆ alkyl, C₃-C₈ cycloalkyl, halogen, —CN, —OR^{a}, optionally substituted phenyl, optionally substituted heteroaryl, optionally substituted heterocyclyl, —C(═O)OR^{a}, —OC(═O)R^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}); and
each occurrence of R^{a} is independently H, optionally substituted C₁-C₆ alkyl, optionally substituted C₃-C₈ cycloalkyl, optionally substituted phenyl, or optionally substituted heteroaryl, or two R^{a} groups combine with the N to which they are bound to form C₃-C₈ heterocyclyl.

36. The method of claim 35, wherein the disease or disorder comprises a neurodegenerative disease or disorder, preferably wherein the neurodegenerative disease or disorder comprises pantothenate kinase-associated neurodegeneration (PKAN).

37. (canceled)

38. The method of claim 35, wherein the disease or disorder comprises an organic aciduria, preferably the disease or disorder comprises methylmalonic acidemia, propionic acidemia, isovaleric acidemia, or maple syrup urine disease.

39. (canceled)

40. The method of claim 35, wherein at least one of the following applies:
(a) the compound is the only therapeutic agent administered to the subject,
(b) the compound is the only therapeutic agent administered to the subject in a sufficient amount to treat or prevent the neurodegenerative disease or disorder or Acyl-CoA dehydrogenation deficiency.

41. (canceled)

42. The method of claim 24, wherein the compound is not: 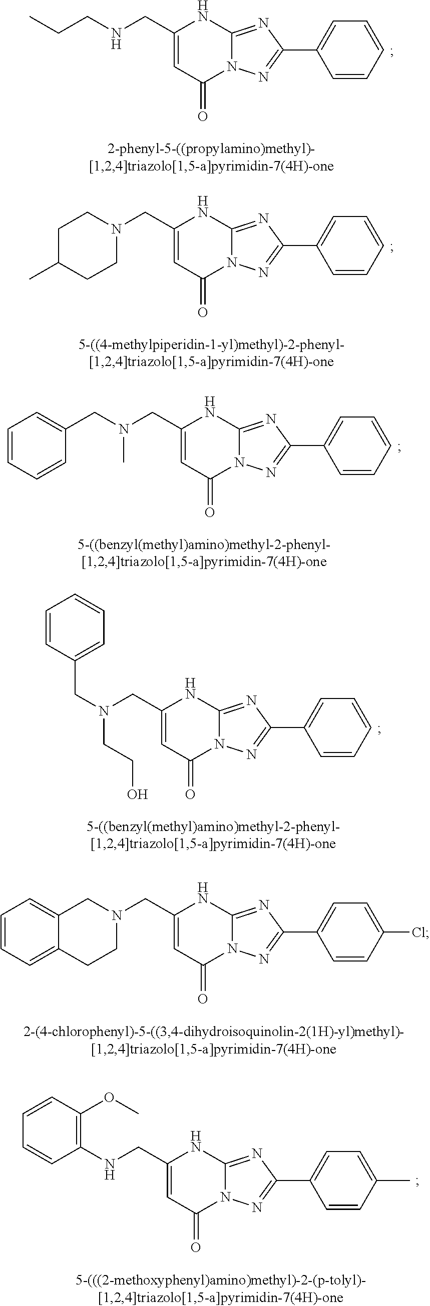 [SMILES: CCCNCc1cc(=O)n2nc(-c3ccccc3)nc2[nH]1 ; CC1CCN(Cc2cc(=O)n3nc(-c4ccccc4)nc3[nH]2)CC1 ; CN(Cc1ccccc1)Cc1cc(=O)n2nc(-c3ccccc3)nc2[nH]1 ; O=c1cc(CN(CCO)Cc2ccccc2)[nH]c2nc(-c3ccccc3)nn12 ; O=c1cc(CN2CCc3ccccc3C2)[nH]c2nc(-c3ccc(Cl)cc3)nn12 ; COc1ccccc1NCc1cc(=O)n2nc(-c3ccc(C)cc3)nc2[nH]1] 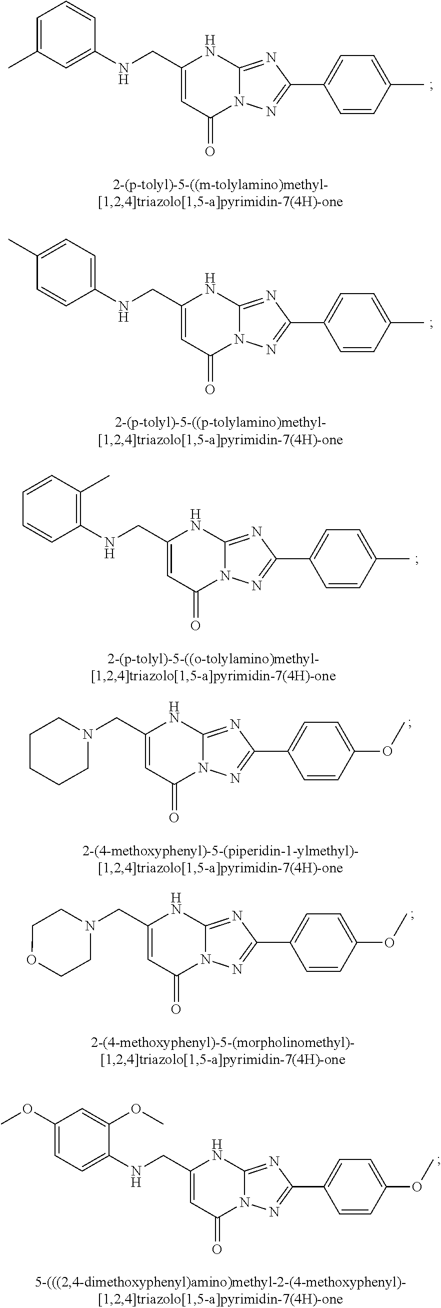 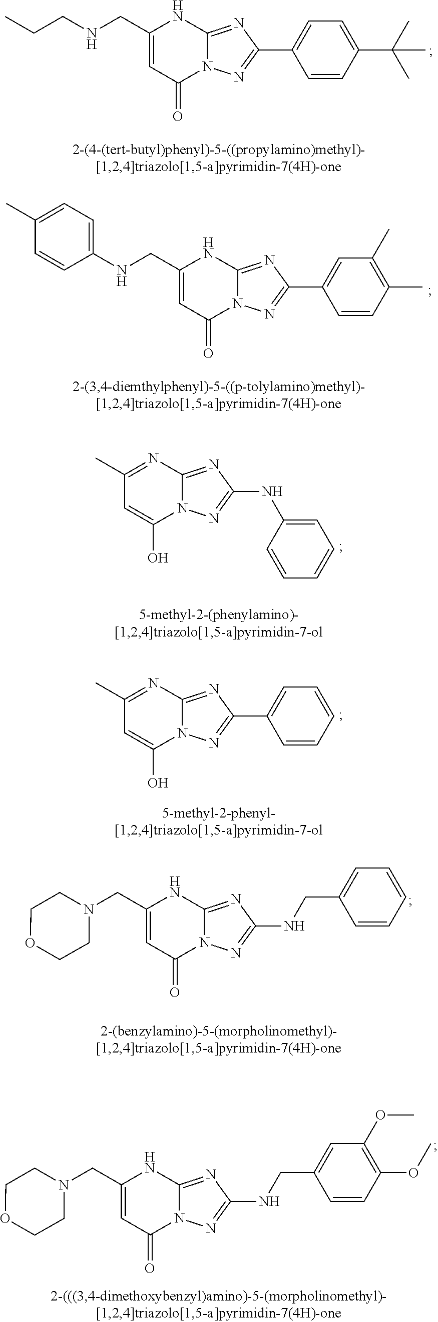 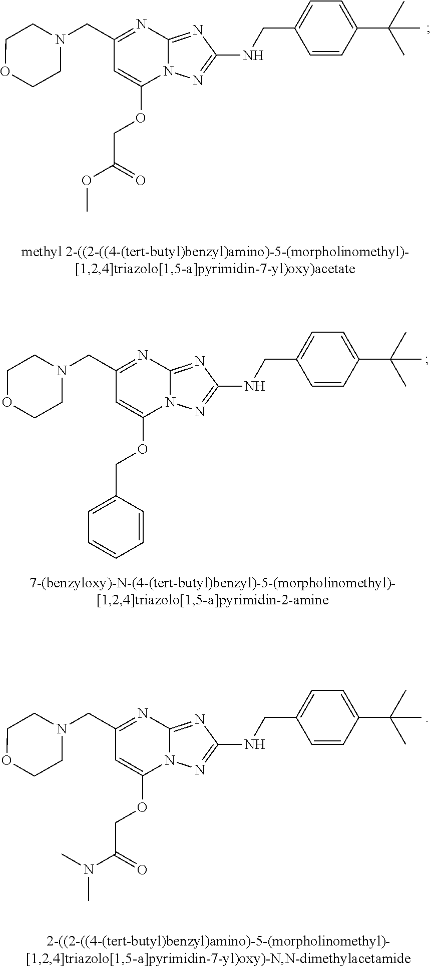 [SMILES: COC(=O)COc1cc(CN2CCOCC2)nc2nc(NCc3ccc(C(C)(C)C)cc3)nn12 ; CC(C)(C)c1ccc(CNc2nc3nc(CN4CCOCC4)cc(OCc4ccccc4)n3n2)cc1 ; CN(C)C(=O)COc1cc(CN2CCOCC2)nc2nc(NCc3ccc(C(C)(C)C)cc3)nn12]
